(19) United States
(12) Patent Application Publication
(10) Pub. No.: US 2023/0043356 A1
(43) Pub. Date: Feb. 9, 2023

(54) A SOLVENT DRYING COMPOSITION AND PROCESSES THEREFOR

(71) Applicant: Aquafortus Technologies Limited, Auckland (NZ)

(72) Inventors: Chaitra PRAKASH, Auckland (NZ); Haiming TANG, Auckland (NZ)

(21) Appl. No.: 17/599,284

(22) PCT Filed: Apr. 2, 2020
(86) PCT No.: PCT/NZ2020/050034

§ 371 Date: Sep. 28, 2021

Related U.S. Application Data
(60) Provisional application No. 62/867,488, filed on Jun. 27, 2019.
(60) Provisional application No. 62/828,607, filed on Apr. 3, 2019.
(60) Provisional application No. 62/828,668, filed on Apr. 3, 2019.

(51) Int. Cl. B01D 11/04; B01J 14/00; C07C 45/80

(52) U.S. Cl. CPC B01D 11/0492; B01J 14/00; C07C 45/80

(57) ABSTRACT

The present disclosure relates to a solvent drying composition and processes therefor. The present disclosure more specifically relates to a solvent drying composition that in use releases water from a solvent mixture. The present disclosure also relates to a process for recovering a solvent drying composition, more specifically to a process for recovering a solvent drying composition used in an osmotic process.

### FIELD OF THE INVENTION

This application is a 35 U.S.C. 371 national stage filing of International Application No. PCT/NZ2020/050034, filed on Apr. 2, 2020, which claims priority to U.S. Provisional Application No. 62/828,607, filed on Apr. 3, 2019, U.S. Provisional Application No. 62/828,668, filed on Apr. 3, 2019, and U.S. Provisional Application No. 62/867,488, filed on Jun. 27, 2019. The entire contents of the aforementioned applications are hereby incorporated by reference.

### BACKGROUND OF THE INVENTION

The extraction of water or drying of water from solvent mixtures is typically a high energy and time-consuming task.

Jessop et. al. in U.S. 2014/0076810 describe a reversible water or aqueous solution and its use. The reversible water or aqueous solution is formed by adding an ionisable additive comprising an ionisable functional group having at least one nitrogen atom. The additive is further described as a monoamine, a diamine, a triamine, a tetramine or a polyamine, such as a polymer or a biopolymer. The reversible water or aqueous solution is capable of reversibly switching between an initial ionic strength and an increased ionic strength by using a trigger, such as bubbling with CO₂, CS₂ or COS or treatment with a Bronsted acid such as formic acid, hydrochloric acid, sulphuric acid or carbonic acid. To enable this reversibility the ionic form of the additive should be capable of deprotonation through the action of the ionising trigger. This necessarily requires a reversible interaction between the ionic form of the trigger and the additive as shown in FIG. 1 of Jessop. The reversibility of the water or aqueous solution allows for the control of solubility or insolubility of various hydrophobic liquids or solvents in the water or aqueous solution. This provides a means of separating moderately hydrophobic solvents from the switchable water. However, one of the difficulties with the Jessop work is that is difficult to disassociate the CO₂ from the amine to achieve the reversible water. Trace amounts of CO₂ and amine can remain solubilised in the draw solution and heating, stripping and the kinetics of recovery are slow, energy intensive in the of the order of hours to minutes.

It is an object of the present invention to provide a solvent drying composition that overcomes these difficulties or to at least provide a useful alternative.

### SUMMARY OF THE INVENTION

In a first aspect, the present invention provides a solvent drying composition for use in recovering water from a solvent, the composition comprising a complex of:

a) at least one amine or ammonium salt containing compound and

b) at least one carboxylic acid containing compound or an alkylsulfonic acid; or a combination thereof,

wherein in use the water is released from the solvent upon migration of the composition through the solvent, the released water forming an immiscible aqueous layer with the solvent.

In a second aspect, the present invention provides a solvent drying composition, the composition comprising of:

a) a complex of at least one amine or ammonium salt containing compound and

at least one carboxylic acid containing compound or an alkylsulfonic acid; or a combination thereof, in a solvent comprising

b) at least one amine containing compound at least one enolisable carbonyl and water,

wherein in use water in the solvent is released to form an immiscible aqueous layer with the solvent drying composition.

In one embodiment the carboxylic acid containing compound is selected from one or more of the following:

a) a compound of Formula I,

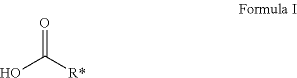

wherein R* is selected from, -C₁-C₇ alkyl-OH, -C₁-C₇ alkyl, -C₁-C₇ alkyl-NH₂, -C₁-C₇ alkyl-NHR₃ and -C₁-C₇ alkyl NR₃R₄, wherein each R₃ and R₄ are selected from —H, —OH, -halo, -C₁-C₇ alkyl, -C₁-C₇ alkyl-OH, —C(O)OH, —C(O)—H, or —C(O)-(C₁-C₇ alkyl); and

b) a polymer containing one or more carboxylic acid groups.

In one embodiment the alkylsulfonic acid is isoethionic acid.

In another embodiment the solvent comprises at least a secondary or tertiary amine or a combination thereof.

In one embodiment the solvent comprises at least one enolisable carbonyl of Formula II,

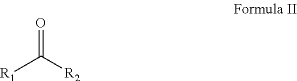

wherein

a) R₁ and R₂ are independently selected from a -C₁-C₇ alkyl or a -C₃-C₇ monocyclic; or

b) one of R₁ or R₂ is selected from a —O-(C₁-C₇ alkyl) and the other is selected from a -C₁-C₇ alkyl, or

c) R₁ and R₂ together, with the carbonyl of Formula II, form a 3-15 membered monocyclic ketone or a 3-15 membered monocyclic heterocyclic ketone.

In one embodiment the carboxylic containing compound of Formula I is selected from acetic acid, citric acid and glycolic acid or a combination thereof.

In one embodiment the molar ratio of the at least amine or ammonium salt containing compound to the at least one carboxylic acid containing compound or an alkylsulfonic acid or a combination thereof; is selected from about 1:99 or 99:1; or about 1:50 or 50:1; or about 1:10 or 10:1; or about 1:5 or 5:1; or about 1:3 or 3:1; or about 1:2 or 2:1; or about 1:1.

In a third aspect, the present invention provides a solvent drying composition, the composition comprising:

a) a complex of at least one amine or ammonium salt containing compound and

b) at least one carboxylic acid containing a compound of Formula I,

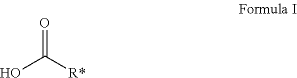

wherein R* is selected from, -C₁-C₇ alkyl-OH, -C₁-C₇ alkyl, -C₁-C₇ alkyl-NH₂, -C₁-C₇ alkyl-NHR₃ and -C₁-C₇ alkyl NR₃R₄, wherein each R₃ and R₄ are selected from -H, —OH, -halo, -C₁-C₇ alkyl, -C₁-C₇ alkyl-OH, —C(O)OH, —C(O)—H, or —C(O)-(C₁-C₇ alkyl); or an alkylsulfonic acid; or a combination thereof; in a solvent comprising

c) at least one amine containing compound, at least one enolisable carbonyl and water,

wherein in use the water in the solvent is released to form an immiscible aqueous layer with the solvent drying composition.

In one embodiment the complex of the at least one amine or ammonium salt containing compound and the at least one carboxylic acid containing compound of Formula 1 is irreversibly protonated.

In another embodiment the solvent comprises at least a secondary or tertiary amine or a combination thereof.

In one embodiment the solvent comprises at least one enolisable carbonyl of Formula II,

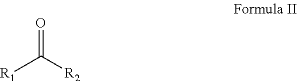

wherein

d) R₁ and R₂ are independently selected from a -C₁-C₇ alkyl or a -C₃-C₇ monocyclic; or

e) one of R₁ or R₂ is selected from a —O-(C₁-C₇ alkyl) and the other is selected from a -C₁-C₇ alkyl, or

f) R₁ and R₂ together, with the carbonyl of Formula II, form a 3-15 membered monocyclic ketone or a 3-15 membered monocyclic heterocyclic ketone.

In one embodiment the -carboxylic acid containing compound of Formula I is selected from acetic acid, citric acid and glycolic acid or a combination thereof.

In one embodiment the alkylsulfonic acid is isoethionic acid.

In one embodiment the complex of the at least one amine or ammonium salt containing compound and the at least one carboxylic acid containing compound of Formula I is irreversibly protonated.

In one embodiment the molar ratio of the at least amine or ammonium salt containing compound to the at least one carboxylic acid containing compound or an alkylsulfonic acid or a combination thereof; is selected from about 1:99 or 99:1; or about 1:50 or 50:1; or about 1:10 or 10:1; or about 1:5 or 5:1; or about 1:3 or 3:1; or about 1:2 or 2:1; or about 1:1.

In a fourth aspect, the present invention provides a complex composition wherein the complex comprises at least one amine or ammonium salt containing compound and at least one carboxylic acid containing compound selected from one or more of the following:

a) compound of Formula I,

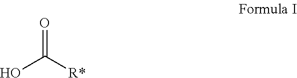

wherein R* is selected from, -C₁-C₇ alkyl-OH, -C₁-C₇ alkyl, -C₁-C₇ alkyl-NH₂, -C₁-C₇ alkyl-NHR₃ and -C₁-C₇ alkyl NR₃R₄, wherein each R₃ and R₄ are selected from —H, —OH, -halo, -C₁-C₇ alkyl, -C₁-C₇ alkyl-OH, —C(O)OH, —C(O)—H, or —C(O)-(C₁-C₇ alkyl);

b) a polymer containing one or more carboxylic acid groups; or an alkylsulfonic acid; or a combination thereof

the complex being suitable for use in recovering water from a solvent, wherein water is released from the solvent upon migration of the composition through the solvent, the released water forming an immiscible aqueous layer with the solvent

and wherein the solvent comprises:
a) at least one amine containing compound,
b) at least one enolisable carbonyl, and
c) water.

In another embodiment the solvent comprises at least a secondary or tertiary amine or a combination thereof.

In one embodiment the solvent comprises at least one enolisable carbonyl of Formula II,

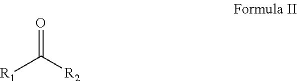

wherein

a) R₁ and R₂ are independently selected from a -C₁-C₇ alkyl or a -C₃-C₇ monocyclic; or

b) one of R₁ or R₂ is selected from a —O-(C₁-C₇ alkyl) and the other is selected from a -C₁-C₇ alkyl, or

c) R₁ and R₂ together, with the carbonyl of Formula II, form a 3-15 membered monocyclic ketone or a 3-15 membered monocyclic heterocyclic ketone.

In one embodiment the at least one amine containing compound of the complex is a secondary or tertiary amine or combination thereof.

In one embodiment the carboxylic acid containing compound of Formula I is selected from acetic acid, citric acid and glycolic acid or a combination thereof.

In one embodiment the alkylsulfonic acid is isoethionic acid.

In one embodiment the molar ratio of the at least amine or ammonium salt containing compound to the at least one carboxylic acid containing compound or an alkylsulfonic acid or a combination thereof; is selected from about 1:99 or 99:1; or about 1:50 or 50:1; or about 1:10 or 10:1; or about 1:5 or 5:1; or about 1:3 or 3:1; or about 1:2 or 2:1; or about 1:1.

In one embodiment the complex of the at least one amine or ammonium salt containing compound and the at least one carboxylic acid containing compound of Formula I is irreversibly protonated.

In a fifth aspect, the present invention provides a method of recovering water from a solvent, the method including the steps of contacting the solvent drying composition for use in recovering water from a solvent, the composition comprising a complex of:

a) at least one amine or ammonium salt containing compound and

b) at least one carboxylic acid containing compound, or an alkylsulfonic acid; or a combination thereof;

and allowing the migration of the complex composition through the solvent, whereupon the water is released from the solvent forming an immiscible aqueous layer with the solvent.

In one embodiment method includes the step of separating the recovered water from the immiscible solvent layer.

In one embodiment the solvent comprises:

a) at least one amine containing compound,

b) at least one enolisable carbonyl.

In a sixth aspect, the present invention provides a method of recovering water from a solvent, the method including the steps of contacting the solvent drying composition for use in recovering water from a solvent, the composition comprising

a) at least one amine containing compound,

b) at least one enolisable carbonyl.

contacting the solvent with a complex composition wherein the complex comprises at least one amine or ammonium salt containing compound and at least:
(a) an alkylsulfonic acid; or
(b) at least one carboxylic acid containing compound of Formula I,

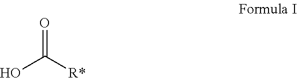

wherein R* is selected from, -C₁-C₇ alkyl-OH, -C₁-C₇ alkyl, -C₁-C₇ alkyl-NH₂, -C₁-C₇ alkyl-NHR₃ and -C₁-C₇ alkyl NR₃R₄, wherein each R₃ and R₄ are selected from -H, -OH, -halo, -C₁-C₇ alkyl, -C₁-C₇ alkyl-OH, —C(O)OH, —C(O)—H, or —C(O)-(C₁-C₇ alkyl); or
(c) a combination thereof; and
   allowing the migration of the complex composition through the solvent, whereupon the water is released from the solvent forming an immiscible aqueous layer with the solvent.

In one embodiment method includes the step of separating the recovered water from the immiscible solvent layer.

In one embodiment the solvent comprises:

a) at least one amine containing compound,

b) at least one enolisable carbonyl.

In another aspect, the present invention provides a process for using a solvent drying composition as defined above to recover water from a solvent, the composition comprising a complex of:

a) at least one amine or ammonium salt containing compound and

b) at least one carboxylic acid containing compound or an alkylsulfonic acid; or a combination thereof,

wherein in use the water is released from the solvent upon migration of the composition through the solvent, the released water forming an immiscible aqueous layer with the solvent; the process comprising the steps of:

1) bringing the solvent drying composition into contact with the solvent to release the water from the solvent upon migration of the composition through the solvent, the released water and solvent drying composition forming an immiscible aqueous layer with the solvent, and

2) recovering the solvent drying composition from the immiscible aqueous layer.

In one embodiment the process includes the step of recovering the solvent.

In one embodiment the recovered solvent drying composition is recycled for use in a further solvent drying process. In a preferred embodiment the process of recovering the solvent drying composition is a continuous recovery process.

In one embodiment the step of recovering the solvent drying solution is achieved by one or more of the following techniques, membrane distillation, pervaporation, osmosis, pressure driven membrane processes, osmotically driven membrane processes, osmotically assisted pressure driven membrane processes, pressure assisted osmotically driven membrane processes, filtration, mechanical vapor recompression, evaporation based processes, water specific reactant, or crystallisation techniques or the like.

In one embodiment the step of recovering the solvent drying solution is achieved by a pressure assisted osmosis technique.

In one embodiment the at least one carboxylic acid containing compound is selected from one or more of the following:

a) a compound of Formula I,

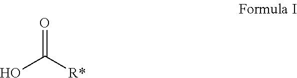

wherein R* is selected from, -C₁-C₇ alkyl-OH, -C₁-C₇ alkyl, -C₁-C₇ alkyl-NH₂, -C₁-C₇ alkyl-NHR³ and -C₁-C₇ alkyl NR³R⁴, wherein each R³ and R⁴ are selected from —H, —OH, -halo, -C₁-C₇ alkyl, -C₁-C₇ alkyl-OH, —C(O)OH, —C(O)—H, or —C(O)-(C₁-C₇ alkyl); and

b) a polymer containing one or more carboxylic acid groups.

In one embodiment the alkylsulfonic acid is isoethionic acid.

In one embodiment the -carboxylic containing compound of Formula I is selected from acetic acid, citric acid and glycolic acid or a combination thereof.

In one embodiment the molar ratio of the at least amine or ammonium salt containing compound to the at least one carboxylic acid containing compound or an alkylsulfonic acid or a combination thereof; is selected from about 1:99 or 99:1; or about 1:50 or 50:1; or about 1:10 or 10:1; or about 1:5 or 5:1; or about 1:3 or 3:1; or about 1:2 or 2:1; or about 1:1.

In another embodiment the at least one amine containing compound is a secondary or tertiary amine or a combination thereof.

In one embodiment the carboxylic acid containing compound is a metal salt-carboxylic acid complex.

In one embodiment the metal salt-carboxylic acid complex is selected from one or more of the following: metal salts having a valency of less than 6, 4 such as Na salts, Fe (II) salts, Fe (III) salts, Cu (II) salts, Al(II) salts, Al(III) salts, Sr (II) salts, Li salts and Ag salts. In one embodiment the metal salts have a valency of less than 4.

In one embodiment the -carboxylic acid containing compound of Formula I is selected from acetic acid, citric acid and glycolic acid or a combination thereof.

In one embodiment the complex comprising:

a) at least one amine or ammonium salt containing compound and

b) at least one carboxylic acid containing compound or an alkylsulfonic acid; or a combination thereof,

is irreversibly protonated.

In one embodiment the solvent is the solvent from which the water is recovered comprises at least one amine containing compound and at least one enolisable carbonyl.

In another embodiment the solvent comprises at least a secondary or tertiary amine or a combination thereof.

In one embodiment the solvent comprises at least one enolisable carbonyl of Formula II,

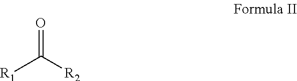

wherein

a) R₁ and R₂ are independently selected from a -C₁-C₇ alkyl or a -C₃-C₇ monocyclic; or

b) one of R₁ or R₂ is selected from a —O-(C₁-C₇ alkyl) and the other is selected from a -C₁-C₇ alkyl, or

c) R₁ and R₂ together, with the carbonyl of Formula II, form a 3-15 membered monocyclic ketone or a 3-15 membered monocyclic heterocyclic ketone or acetophenone.

The foregoing brief summary broadly describes the features and technical advantages of certain embodiments of the present invention. Further technical advantages will be described in the detailed description of the invention and examples that follows.

Novel features that are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to limit the invention's scope.

### BRIEF DESCRIPTION OF THE DRAWINGS

FIG. 1: shows a calibration curve of ethylpiperidine concentration at lower concentrations.

FIG. 2 shows the drying capacity of various amine/acid complexes compared to that of the prior art.

FIG. 3 shows the drying capacity of various amine/amino acid complexes.

FIG. 4 schematically shows a quintuple counter current regeneration process using a commercial brine.

FIG. 5 shows a plot of the various water contents in each stage of the counter current regeneration process outlined in FIG. 4

FIG. 6: shows schematically a process diagram for a pressure assisted osmotic process to recover a solvent drying composition.

FIG. 7 shows a process diagram for a continuous process system for recovering a solvent drying composition.

FIG. 8: shows a graph of the reverse osmosis flux (LMH) data and the rejection % data of 20% (by vol.) diluted drying solvent solution at 60 bar.

FIG. 9: shows the flux data results obtained from 5 different membranes at different pressures.

FIG. 10: shows the rejection % results obtained from 5 different membranes at different pressures.

FIG. 11: shows a process diagram for recovering a solvent drying composition using an electrostatic coalescer.

### DETAILED DESCRIPTION OF THE INVENTION

The following description sets forth numerous exemplary configurations, parameters, and the like. It should be recognised, however, that such description is not intended as a limitation on the scope of the present invention but is instead provided as a description of exemplary embodiments.

### Definitions

In each instance herein, in descriptions, embodiments, and examples of the present invention, the terms “comprising”, “including”, etc., are to be read expansively, without limitation.

Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “about” or “approximately” usually means within 20%, more preferably within 10%, and most preferably still within 5% of a given value or range. Alternatively, the term “about” means within a log (i.e., an order of magnitude) preferably within a factor of two of a given value.

As used herein, the term “at least one amine or ammonium salt containing compound” means any compound that includes an —NH₃, —NHR³ or —NR³R⁴ group or an ammonium salt of —NH₄⁺ with the proviso that ammonium bicarbonate is excluded, wherein each R³ and R⁴ are selected from —H, —OH, -halo, -C₁-C₇ alkyl, -C₁-C₇ alkyl-OH, —C(O)OH, —C(O)—H, or —C(O)-(C₁-C₇ alkyl);

As used herein, the term “carboxylic acid containing compound” is any compound having an —COOH group or a salt thereof, including polymeric compounds, such as polyacrylic acid, copolymers such as poly(acrylic acid-co-maleic acid) solution and the like.

As used herein, the term “alkylsulfonic acid” includes any compound having a R—S(O)₂OH functional group or a salt thereof, where R is a C₁-C₇ alkyl, wherein C₁-C₇ alkyl is as defined below.

As used herein, the term “C₁-C₇ alkyl” refers to a fully saturated branched or unbranched hydrocarbon moiety, which may be a straight or a branched chain of a particular range of 1-7 carbons. Preferably the alkyl comprises 1 to 7 carbon atoms, or 1 to 4 carbon atoms. Representative examples of C₁-C₇alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, and the like. For example, the expression C₁-C₄-alkyl includes, but is not limited to, methyl, ethyl, propyl, butyl, isopropyl, tert-butyl and isobutyl. In one embodiment the C₁-C₇ alkyl group may be substituted with one or more of the following groups: -halo, —OH, —CN, —NO₂, —CΞCH, —SH, -C₁-C₇ alkyl, -(C₁-C₇ alkyl)-OH, —NH₂, —NH(C₁-C₇ alkyl), —N(C₁-C₇ alkyl)₂, —O (C₁-C₇ alkyl), —C(O)—O(-C₁-C₇ alkyl), —C(O)OH; —C(O)—H, or —C(O)-(C₁-C₇ alkyl).

The term “C₃-C₇ monocyclic” as used herein is a 3-, 4-, 5-, 6-, or 7-membered saturated or unsaturated monocyclic ring. Representative C₃-C₇ monocyclic groups include, but are not limited to, cyclopropyl, cyclobutyl, cyclopentyl, cyclohexyl, phenyl, and cycloheptyl. In one embodiment, the C₃-C₇ monocyclic cycloalkyl group may be substituted with one or more of the following groups: -halo, —OH, —CN, —NO₂, —CΞCH, —SH, -C₁-C₇ alkyl, -(C₁-C₇ alkyl)-OH, —NH₂, —NH(C₁-C₇ alkyl), —N(C₁-C₇ alkyl)₂, —O (C₁-C₇ alkyl), —C(O)—O(-C₁-C₇ alkyl), —C(O)OH; —C(O)—H, or —C(O)-(C₁-C₇ alkyl).

The term “3- to 15-membered monocyclic ketone” refers to a 3- to 15-membered non-aromatic monocyclic ring system containing a ketone functional group. Representative examples of a 3- to 15-membered monocyclic ketone include, but are not limited to cyclopropanone, cyclobutanone, cyclopentanone, cyclohexanone, cycloheptanone, cyclooctanone, cyclononanone, cyclodecanone, cycloundecanone, cyclododecanone, cyclotridecanone; cyclotetradecanone and cyclopentadecanone.

In one embodiment, the 3- to 15-membered monocyclic ketone may be substituted with one or more of the following groups-halo, —OH, —CN, —NO₂, —CΞCH, —SH, -C₁-C₇ alkyl, -(C₁-C₇ alkyl)-OH, —NH₂, —NH(C₁-C₇ alkyl)₂, —O (C₁-C₇ alkyl), —C(O)—O(-C₁-C₇ alkyl), —C(O)OH; —C(O)—H, or —C(O)-(C₁-C₇ alkyl).

The term “3- to 15-membered monocyclic heterocyclic ketone” refers to: (i) a 3- or 4-membered non-aromatic monocyclic cycloalkyl in which 1 of the ring carbon atoms has been replaced with an N, O or S atom; or (ii) a 5- to 15-membered non-aromatic monocyclic cycloalkyl in which 1-4 of the ring carbon atoms have been independently replaced with a N, O or S atom. Representative examples of a 3- to 15-membered monocyclic heterocyclic ketone having one N, O or S atom include, but are not limited to oxiran-2-one, thiiran-2-one, oxetan-2-one, oxetan-3-one, azetidin-3-one, thietan-2-one, thietan-3-one, dihydrofuran-2(3H)-one, dihydrofuran-3(2H)-one, pyrrolidin-3-one, dihydrothiophen-3(2H)-one, dihydrothiophen-2(3H)-one, tetrahydro-2H-pyran-2-one, dihydro-2H-pyran-3(4H)-one, dihydro-2H-pyran-4(3H)-one, piperidin-3-one, piperidin-4-one, tetrahydro-2H-thiopyran-2-one, dihydro-2H-thiopyran-3(4H)-one, dihydro-2H-thiopyran-4(3H)-one, oxepan-2-one, oxepan-3-one, oxepan-4-one, thiepan-2-one, thiepan-3-one, thiepan-4-one, azepan-3-one, azepan-4-one, oxocan-2-one, oxocan-3-one, oxocan-4-one, oxocan-5-one, thiocan-2-one, thiocan-3-one, thiocan-4-one, thiocan-5-one, azocan-3-one, azocan-3-one, azocan-4-one, azocan-5-one, azonan-3-one, azonan-4-one, azonan-5-one, oxonan-2-one, oxonan-3-one, oxonan-4-one, oxonan-5-one, thionan-2-one, thionan-3-one, thionan-4-one, thionan-5-one, oxacycloundecan-2-one, oxacycloundecan-3-one, oxacycloundecan-4-one, oxacycloundecan-5-one, oxacycloundecan-6-one, azacycloundecan-3-one, azacycloundecan-4-one, azacycloundecan-5-one, azacycloundecan-6-one, thiacycloundecan-2-one, thiacycloundecan-3-one, thiacycloundecan-4-one, thiacycloundecan-5-one, thiacycloundecan-6-one, oxacyclododecan-2-one, oxacyclododecan-3-one, oxacyclododecan-4-one, oxacyclododecan-5-one, oxacyclododecan-6-one, oxacyclododecan-7-one, azacyclododecan-3-one, azacyclododecan-4-one, azacyclododecan-5-one, azacyclododecan-6-one, azacyclododecan-7-one, thiacyclododecan-2-one, thiacyclododecan-3-one, thiacyclododecan-4-one, thiacyclododecan-5-one, thiacyclododecan-6-one, thiacyclododecan-7-one, oxacyclotridecan-2-one, oxacyclotridecan-3-one, oxacyclotridecan-4-one, oxacyclotridecan-5-one, oxacyclotridecan-6-one, oxacyclotridecan-7-one, azacyclotridecan-3-one, azacyclotridecan-4-one, azacyclotridecan-5-one, azacyclotridecan-6-one, azacyclotridecan-7-one, thiacyclotridecan-2-one, thiacyclotridecan-3-one, thiacyclotridecan-4-one, thiacyclotridecan-5-one, thiacyclotridecan-6-one, thiacyclotridecan-7-one, oxacyclotetradecan-2-one, oxacyclotetradecan-3-one, oxacyclotetradecan-4-one, oxacyclotetradecan-5-one, oxacyclotetradecan-6-one, oxacyclotetradecan-7-one, oxacyclotetradecan-8-one, azacyclotetradecan-3-one, azacyclotetradecan-4-one, azacyclotetradecan-5-one, azacyclotetradecan-6-one, azacyclotetradecan-7-one, azacyclotetradecan-8-one, thiacyclotetradecan-2-one, thiacyclotetradecan-3-one, thiacyclotetradecan-4-one, thiacyclotetradecan-5-one, thiacyclotetradecan-6-one, thiacyclotetradecan-7-one, thiacyclotetradecan-8-one, oxacyclopentadecan-2-one, oxacyclopentadecan-3-one, oxacyclopentadecan-4-one, oxacyclopentadecan-5-one, oxacyclopentadecan-6-one, oxacyclopentadecan-7-one, oxacyclopentadecan-8-one, azacyclopentadecan-3-one, azacyclopentadecan-4-one, azacyclopentadecan-5-one, azacyclopentadecan-6-one, azacyclopentadecan-7-one, azacyclopentadecan-8-one, thiacyclopentadecan-2-one, thiacyclopentadecan-3-one, thiacyclopentadecan-4-one, thiacyclopentadecan-5-one, thiacyclopentadecan-6-one, thiacyclopentadecan-7-one, thiacyclopentadecan-8-one. In one embodiment, the 3- to 15-membered monocyclic heterocyclic ketone group may be substituted with one or more of the following groups-halo, —OH, —CN, —NO₂, —CΞCH, —SH, -C₁-C₆ lower alkyl, -(C₁-C₇ alkyl)-OH, —NH₂, —NH(C₁-C₇ alkyl), —N(C₁-C₇alkyl)₂, —O (C₁-C₇alkyl), —C(O)—O(-C₁-C₇alkyl), —C(O)OH; —C(O)—H, or —C(O)-(C₁-C₇ alkyl). For the avoidance of doubt, the 3-5 membered monocyclic heterocyclic ketone does not include any amide groups where the ketone enolisable carbonyl group is adjacent a N atom in the cyclic structure.

The term “halo” as used herein refers to —F, —Cl, —Br or —I.

The term “an enolisable carbonyl” means a compound that has one or more carbonyl functional groups and wherein at least one of the carbonyl functional groups has alpha hydrogens (Hα) that may be removed by a base to form an enolate and then an enol as shown in the reaction scheme below.

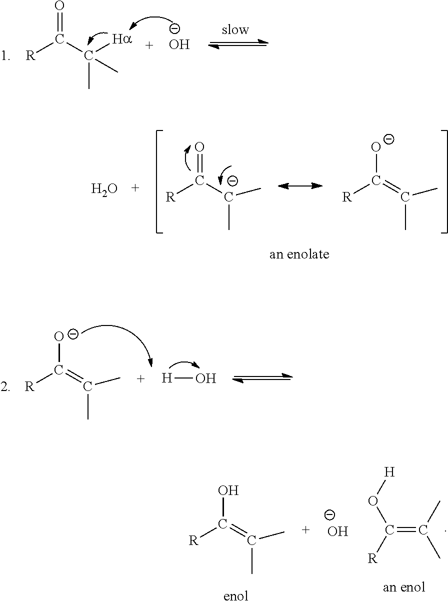

It is to be understood that the term enolisable carbonyl as used in the specification does not include a compound having solely an aldehyde functional group, a compound having solely a carboxylic acid functional group, a compound having solely an amide functional group, a compound having solely an acyl halide functional group or acetylacetone. The enolisable carbonyls of the invention include those exemplified in the specification and without limitation also include the following enolisable carbonyls: 1-acetonapthone, 2-acetonaphthone, 4-methyl-1-acetonaphthone, 1′-hydroxy-2′-acetonaphthone, 2′-hydroxy-1′-acetonaphthone, 2-methoxy-1-acetonaphthone, 4-fluoro-1-acetonapthone; 2-acetylphenanthrene, 3-acetylphenanthrene, 4-acetylphenanthrene, 9-acetylphenanthrene, 6-bromo-9-acetylphenanthrene, 9-fluoro-10-acetylphenanthrene, 9-fluorenone, 9-fluorenone oxime, 2-nitro-9-fluorenone, 3-nitro-9-fluorenone, 4-nitro-9-fluorenone, 2,6-dinitro-9-fluorenone, 2,7-dinitro-9-fluorenone, 2,3,7-trinitro-9-fluorenone, 2-fluoro-9-fluorenone, 1-bromo-9-fluorenone, 2-bromo-9-fluorenone, 2,7-dichloro-9-fluorenone, 2,7-dibromo-9-fluorenone, 2-hydroxy-9-fluorenone, 4-hydroxy-9-fluorenone; 1-methylfluoren-9-one; 4-methylfluoren-9-one; 3,4-dihydro-2(1H)-quinolinone, 7-hydroxy-3,4-dihydro-2(1H)-quinolinone, 6-hydroxy-3,4-dihydro-2(1H)-quinolinone, 8-bromo-2,3-dihydro-4(1H)-quinolinone, 3-butyl-4-hydroxy-1-methyl-2(1H)-quinolinone, 6-fluoro-4,4-dimethyl-3,4-dihydro-2(1H)-quinolinone, 8-fluoro-4,4-dimethyl-3,4-dihydro-2(1H)-quinolinone, 2,6-dimethyl-4(1H)-quinolinone, 3-butyl-4-hydroxy-1-methyl-2(1H)-quinolinone, 1-indanone,5,6-dimethoxy-1-indanone, 6-bromo-1-indanone, 6-methoxy-1-indanone, 2-bromo-1-indanone, 4-bromo-1-indanone, 5-bromo-1-indanone, 5-chloro-1-indanone, 6-chloro-1-indanone, 4,7-dimethyl-1-indanone, 2-methyl-1-indanone, 4-methyl-1-indanone, 5-fluoro-1-indanone, 6-fluoro-1-indanone, 6-(trifluoromethyl)-1-indanone, 4-methoxy-1-indanone, 3,5-dimethoxy-1-indanone, 4,7-dimethoxy-1-indanone, 5-hydroxy-1-indanone, 4-hydroxy-1-indanone, 7-hydroxy-1-indanone, 2-indanone oxime, 2,2-di(methylthio)-1-indanone, (2,4-dimethoxyphenyl)acetone, 3,5-dimethoxyacetophenone, 4-(4-methoxyphenyl)-2-butanone, 3-methoxyphenylacetone, 4-methoxy acetophenone, 4-methoxy-2-phenylacetophenone, 2,5-dimethylphenylacetone, 3,4,5-trimethoxyphenylacetone, 4-hydroxy-3-phenylbutan-2-one, 3-hydroxy-4-phenylbutan-2-one, 3-hydroxy-3-phenylbutan-2-one, 4-hydroxy-4-phenylbutan-2-one, 1-hydroxy-3-phenylbutan-2-one, 3-hydroxy-1-phenylbutan-2-one, 3-hydroxy-1,3-diphenylbutan-2-one, 4-hydroxyphenylacetone, 3,4-dihydroxyphenylacetone, 4-nitrophenylacetone, acetophenone, 4-methyl acetophenone, benzylacetone, 3-methylphenylacetone, 4-methylphenylacetone, 4-ethylphenylacetone, 1-phenylbutan-2-one, 3-phenylbutan-2-one, 4-phenylbutan-2-one, 1-bromo-4-phenylbutan-2-one, 3-methly-1-phenylbutan-2-one, 3-methly-4-phenylbutan-2-one, ethyl phenyl ketone, butyl phenyl ketone, cyclopropyl phenyl ketone, cyclopentyl phenyl ketone, cyclobutyl phenyl ketone, cyclohexyl phenyl ketone, 2-phenylcyclopentanone, 3-phenylcyclopentanone, 2-phenylcyclohexanone, 3-phenylcyclohexanone, 2-phenylcycloheptanone,3-phenylcycloheptanone, 4-chlorophenyl acetone, 4-chloro-2-phenylacetophenone, 2,6-dichlorophenylacetone, 3-chlorophenylacetone, 2,6-difluorophenylacetone, 1-bromo-1-phenylbutan-2-one, 3-bromo-4-phenylbutan-2-one, 1-bromo-4-phenylbutan-2-one, 3-chloro-4-phenylbutan-2-one, 2-acetylthiophene, cyclopropyl-2-thienyl ketone, 2-acetylfuran, 2-furyl methyl ketone, 1-acetylpyrrole, 2-acetylpyrrole, 4-methyl-2-phenylacetophenone, 1,3-diphenylacetone, 4,4-diphenylbutan-2-one, benzophenone, 4-napthyl phenyl ketone, 2-benzoylpyridine, 3-benzoylpyridine, 4-benzoylpyridine, 2-(4-chlorobenzoyl) pyridine, 2-benzoylthiophene, 2-benzoylpyrrole, di(3-thiophenyl) methanone, 3-phenyl-1-(2-thienyl)-2-propen-1-one, and piperonyl acetone.

The term “amine containing compound, includes any compound that includes one or more amine functionalities, but does not include a heterocyclic amine where the heterocyclic ring includes an oxygen or sulphur atom as well as at least one amine group; such as for example 4-ethylmorpholine.

The term “tertiary amine containing compound” preferably means a compound having at least one tertiary amine group, but it is to be appreciated that the compound may have more than one tertiary amine group or further may be a mixture of tertiary amine containing compounds. Preferably the tertiary amine containing compound is a base, such as a Lewis base. If the base is a Lewis base, it is envisaged that a Lewis adduct may be formed with the enolisable carbonyl. In one embodiment it is preferred that the tertiary amine containing compound is immiscible with water at or above 20 degrees Celsius under one standard atmosphere of pressure. The solution may include a combination of more than one tertiary amine containing compound. The tertiary amine containing compound may be aliphatic, conjugated, asymmetric or cyclic or a combination thereof.

Examples of suitable tertiary amine containing compounds include the following:

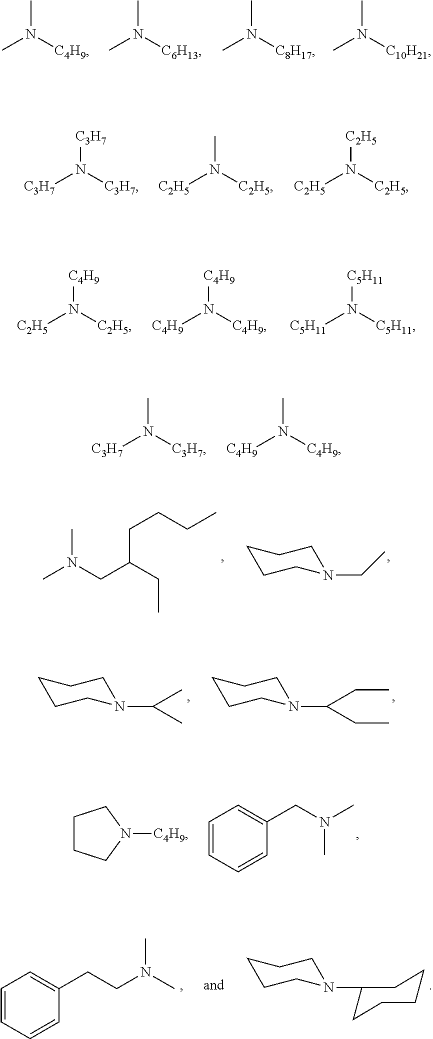

In one embodiment the tertiary amine containing compound is selected from 1-ethylpyrrolidine, ethylpiperidine, 2-methylpyridine and N-methylpiperidine.

In one embodiment the tertiary amine containing compound is selected from a —N(C₁-C₇ alkyl)₃. In another embodiment the tertiary amine containing compound is selected from a —N(C₁-C₄ alkyl)₃. In yet a further embodiment the tertiary amine containing compound is —N(C₂ alkyl)₃ (triethylamine).

It will be appreciated that the above listed tertiary amine containing compounds are simple enough for production on an industrial scale.

It is to be appreciated that the molar ratio of the at least one tertiary amine containing compound to the one or more enolisable carbonyls of Formula II may be present in a number of molar ratios including of about 1:99 or 99:1; of about 1:50 or 50:1; of about 1:10 or 10:1; of about 1:5 or 5:1; of about 1:3 or 3:1; of about 1:2 or 2:1 or of about 1:1.

It is to be appreciated that the molar ratio of the at least amine or ammonium salt containing compound to the at least one carboxylic acid containing compound or an alkylsulfonic acid or a combination thereof; is selected from about 1:99 or 99:1; or about 1:50 or 50:1; or about 1:10 or 10:1; or about 1:5 or 5:1; or about 1:3 or 3:1; or about 1:2 or 2:1; or about 1:1.

### EXAMPLES

The examples described herein are provided for the purpose of illustrating specific embodiments of the invention and are not intended to limit the invention in any way. Persons of ordinary skill can utilise the disclosures and teachings herein to produce other embodiments and variations without undue experimentation. All such embodiments and variations are considered to be part of this invention.

### Preparative Examples

Preparation Example 1.—Water Absorbing Solvent Mixture Solution

Preparation of a water absorbing solvent mixture for testing purposes. The following method was employed to generate a standard water absorbing solvent mixture solution.

1. Commercially available, analytical grade 2-butanone (also known as methylethyl ketone MEK) and triethylamine (TEA) was mixed in a 2:1 molar ratio as follows in Table 1 to create the water absorbing solvent mixture in its “dry” state (without water):

TABLE 1

| Total quantity | | |
|---|---|---|
| of solvent | | |
| mixture made | 2-Butanone | Triethylamine |
| (L) | (L) | (L) |
| 1 | 0.563 | 0.437 |
| 2 | 1.125 | 0.875 |
| 5 | 2.813 | 2.187 |
| 10 | 5.626 | 4.374 |
| 20 | 11.253 | 8.747 |

2. 10% deionised water was added to the solvent mixture in the amounts shown below in Table 2 and well shaken. The addition of water to the solvent mixture created a “wet solvent mixture”.

TABLE 2

| Volume | Volume |
|---|---|
| of solvent | of water |
| mixture | to add |
| (L) | (mL) |
| 1 | 100 |
| 2 | 200 |
| 5 | 500 |
| 10 | 1000 |
| 20 | 2000 |

3. Once the wet solvent mixture had been prepared, various complexes of [amine*+carboxylic acid containing compound]-could be studied as drying agents, ie agents for removing water from the solvent mixture. This would involve adding the selected drying agent to the wet solvent mixture with vigorous shaking. The drying agent was added at a water:drying agent ratio of 2:1 as shown in Table 3.

TABLE 3

| | Volume | Volume |
|---|---|---|
| | of DI water | of Drying |
| Volume | added to | Agent to |
| of solvent | solvent | add (mL) to |
| mixture | mixture | wet solvent |
| (L) | (mL) | mixture |
| 1 | 100 | 50 |
| 2 | 200 | 100 |
| 5 | 500 | 250 |
| 10 | 1000 | 500 |
| 20 | 2000 | 1000 |

4. The two liquids were allowed to fully separate.

5. The drying agent (bottom layer) was decanted and disposed of.

6. A Standard Addition test (in triplicate) was carried out to calculate the concentration of water in the sample, using a gas chromatogram.

All GC data was collected on a SHIMADZU Nexis 2030 gas chromatograph fitted with a SUPELCO WATERCOL 1910 column. The GC parameters were set up as shown below:

| Parameter | Setting |
|---|---|
| Injection Volume | 1.0 μL |
| Injection temperature | 250° C. |
| Injection mode | Split |
| Split ratio | 100.0 |
| Carrier gas | He |
| Carrier gas pressure | 53.1 kPa |
| Column flow | 0.93 mL/min |
| Liner velocity | 22.0 cm/s |
| Column length | 30.0 m |
| Column inner diameter | 0.32 |
| Column method | Isocratic |
| Column temperature | 163.0° C. |
| Total time | 9 min |
| Detector | TCD |
| TCD sample rate | 40 ms |
| TCD current | 70 mA |
| Makeup gas | He |
| Makeup flow | 8.0 mL/min |
| TCD temperature | 200° C. |

Column Method:

| Rate | Temperature | Hold Time |
|---|---|---|
| (° C./min) | (° C.) | (min) |
| | 100.00 | 2.55 |
| 25.0 | 168.0 | 5.0 |

Total program time 10:27 min

Preparative Example 2.—Drying Agent Complex

The drying agent complex was made up to a molar ratio of 1:1 of citric acid: ethylpiperidine 10% excess citric acid was then added to ensure that all the ethylpiperidine had complexed to form the complex [amine*+carboxylic acid containing compound] to remove any chance of “free” ethylpiperidine.

### Example 1—The Water Absorbency of Various Complexes [amine*+Carboxylic Acid containing Compound]

Several complexes of amine with citric acid or amine with glycolic acid were evaluated for regenerant capabilities. The complexes of citric acid and glycolic acid were prepared at the same molality of 6.9 mol/kg. Various combinations of solvent mixtures were prepared as outlined in Table 4 below for reaction with either 6.9 mol/kg of citric acid or glycolic acid to form the various complexes [amine*+carboxylic acid containing compound] which were then be tested for water absorbing capabilities:

TABLE 4
The composition of the various solvent mixtures

| Various solvent mixtures | Molar ratio |
|---|---|
| Triethylamine:MEK | 0.5:1 |
| Ethylpiperidine:MEK | 0.5:1 |
| (Triethylamine:Ethylpiperidine):MEK | (0.3:0.2):1 |

The complexes [amine*+carboxylic acid containing compound] obtained were tested for their water recovery capabilities by the following procedures: 0.2 ml of the various complexes were each added to 20 ml of the wet solvent mixture (prepared in accordance with Preparative Example 1 above).
The resulting mixture was mixed by a Vortex mixer for 30 seconds and then separated by the centrifuge fitted with a 130 mm diameter 4 arm swing rotor at 4000 rpm for 60 seconds.
The remaining water in the solvent mixture was measured using gas chromatography through the method of standard addition.

The results obtained are tabulated in table 5.

TABLE 5
Composition of new amine/acid complex combination (by contacting acid + amine
complex with wet solvent mixture) and their water absorbing capabilities.

| | | | | | | | | H2O in | | H₂O in |
|---|---|---|---|---|---|---|---|---|---|---|
| | | | | | | Ratio of | Wet | Wet | | Dry |
| | Acid | | Distilled | The | | Amine | solvent | solvent | | solvent |
| | weight | Moles | water | molality | | toMEK | mixture | mixture | Complex | mixture |
| Acid | (g) | (mol) | (g) | mol/kg | Amine | (X:1) | used (mL) | % | used (mL) | % |
| Glycolic | 5.24 | 0.069 | 10.0 | 6.90 | TEA | 0.5 | 20.0 | 7.10 | 0.2 | 6.00 |
| Glycolic | 5.24 | 0.069 | 10.0 | 6.90 | EP | 0.5 | 20.0 | 7.10 | 0.2 | 6.00 |
| Glycolic | 5.24 | 0.069 | 10.0 | 6.90 | TEA | 0.3:0.2 | 20.0 | 7.10 | 0.2 | 5.90 |
| | | | | | and | | | | | |
| | | | | | EP | | | | | |
| Citric | 13.2 | 0.069 | 10.0 | 6.90 | TEA | 0.5 | 20.0 | 7.10 | 0.2 | 5.20 |
| acid | | | | | | | | | | |
| Citric | 13.2 | 0.0690 | 10.0 | 6.90 | EP | 0.5 | 20.0 | 7.10 | 0.2 | 5.30 |
| Citric | 13.2 | 0.0690 | 10.0 | 6.90 | TEA | 0.3:0.2 | 20.0 | 7.10 | 0.2 | 5.30 |
| | | | | | and | | | | | |
| | | | | | EP | | | | | |
| Citric | 13.2 | 0.0690 | 10.0 | 6.90 | IBA | 0.5 | 20 | 7.10 | 0.2 | 6 |
| Citric | 13.2 | 0.0690 | 10.0 | 6.90 | PYR | 0.5 | 20 | 7.10 | 0.2 | 5.8 |

TEA = triethylamine;
EP = ethylpiperidine;
IBA = isobutylamine;
PYR = pyrrolidine

The results shown in Table 5 demonstrate that amine/acid salts also exhibit regen water-absorbing abilities, hence can function as a regenerant as well. With the same concentration, the water-absorbing ability of citric salts was better than glycolic salts.

### Example 1 continued—The Water Absorbency of Various Complexes [Ammonium Salt+Carboxylic Acid containing Compound]

Complexes of ammonium salts with citric acid were evaluated for water recovery capabilities. Dry and wet solvent mixtures were prepared as per preparative example 1 above.

Ammonium citrate was prepared as follows:
Citric acid (13.96 g, 0.073 mol) was added to 10 ml of wt 28% ammonia in water
(NH4OH: 2.55 g, 0.073 mol).
   The mixture was stirred at room temperature for 30 mins.

The preparation of saturated solution is below as follows:
A quantity of acids was added to 10 m L of distilled water.
The solution was stirred at the room temperature.
Once no more acid dissolves, the stirring was stopped and the saturated solution was used.

In Table 6, some potential ammonium salt regenerants were made as saturated solutions. The regenerant composition and source are listed in the table. The procedure to measure water absorption capability is as follows:
0.2 ml of the various Regenerants are each added to 20 ml of the wet solvent mixture.
It was mixed by a Vortex mixer for 30 seconds and then separated by the centrifuge fitted with a 130 mm diameter 4 arm swing rotor at 4000 rpm for 60 seconds.
Remaining water in the solvent mixture was checked by the GC through the method of standard addition.

TABLE 6
Potential ammonium salt regenerants

| | | | Percentage |
|---|---|---|---|
| | | | Concentration |
| | | | of water in the |
| | | Percentage | solvent mixture |
| | | Concentration | after drying with |
| | | of water in wet | the ammonium |
| | | solvent mixture | salt acid complex |
| Ammonium salt | Source of Salts | (%) | (%) |
| Ammonium | Prepared | 7.10 | 4.40 |
| citrate | according | | |
| | to the method | | |
| | above | | |
| Isethionic acid | Sigma-Aldrich | 7.10 | 5.4 |
| ammonium salt | | | |

*Ammonium citrate was prepared as the method above.

A range of carboxylic group containing compounds were tested to determine their water absorbing capacities. As above, wet solvent mixture samples were prepared according to preparative example 1 above. Various carboxylic containing compounds were purchased from Sigma-Aldrich, such as poly(acrylic acid-co-maleic acid) solution, Poly(acrylic acid), glycolic acid and tartaric. The carboxylic acid containing compounds were prepared as shown in Table 6 and Table 7. The samples in Table 6 were diluted in half concentration and used for the tests, which were evaluated in Table 7.

TABLE 7
The table showing the potential acids at the molality of —COOH of 9.80 mol/kg

| | | | Number of | Moles of | Moles of | | The |
|---|---|---|---|---|---|---|---|
| Potential | Weight | MW of the | —COOH | Potential | —COOH | Distilled | molality of |
| carboxylic | used | Regenerant | Groups in | Regen | group | water | —COOH |
| acid | (g) | (g/mol) | Regenerant | (mol) | (mol) | (kg) | (mol/kg) |
| Poly(acrylic | 10.0 | 3000 | 48.0 | 0.0033 | 0.16 | 0.016 | 9.8 |
| acid-co- | | | | | | | |
| maleic | | | | | | | |
| acid) | | | | | | | |
| solution | | | | | | | |
| Poly(acrylic | 7.20 | 1800 | 25.0 | 0.0040 | 0.098 | 0.010 | 9.8 |
| acid) | | | | | | | |
| solution | | | | | | | |
| Glycolic | 7.45 | 76.1 | 1.00 | 0.098 | 0.098 | 0.010 | 9.8 |
| acid | | | | | | | |
| Tartaric | 7.35 | 150 | 2.00 | 0.049 | 0.098 | 0.010 | 9.8 |
| Acid | | | | | | | |
| Citric acid | 6.34 | 192 | 3.00 | 0.033 | 0.098 | 0.010 | 9.8 |

The molality (mol/kg) of —COOH was calculated using the formula:

$$\frac{Moles of - COOH}{The weight of distilled water}$$

TABLE 8
The table showing the potential carboxylic acids at the molality
of 0.200 mol/kg

| | | MW of the | | | |
|---|---|---|---|---|---|
| Potential | | carboxylic | The moles | Distilled | The |
| carboxylic | weight | acid | of acids | water | molality |
| acid | (g) | (g/mol) | (mol) | (kg) | (mol/kg) |
| poly(acrylic | 10.0 | 3000 | 0.0033 | 0.017 | 0.2 |
| acid-co- | | | | | |
| maleic | | | | | |
| acid) | | | | | |
| solution | | | | | |
| Poly(acrylic | 3.60 | 1800 | 0.0020 | 0.010 | 0.20 |
| acid) | | | | | |
| solution | | | | | |
| glycolic | 0.152 | 76.05 | 0.0020 | 0.010 | 0.2 |
| acid | | | | | |
| tartaric | 0.300 | 150 | 0.0020 | 0.010 | 0.2 |
| Acid | | | | | |
| citric acid | 0.384 | 192 | 0.0020 | 0.010 | 0.2 |

The molality (mol/kg) was calculated using the formula:

$$\frac{Moles of acids}{The weight of distilled water}$$

The following steps were taken to measure the water releasing capabilities for these carboxylic acid group containing candidates:
0.2 ml of each carboxylic acid containing compound was added to 20 ml of the wet solvent mixture.
The resulting combination was mixed by the instrument of mixer for 30 seconds and then separated by the centrifuge fitted with a 130 mm diameter 4 arm swing rotor at 4000 rpm for 60 seconds.
Remaining water in the solvent mixture was checked by the GC through the method of standard addition.

Observation and Analysis:

TABLE 9
The Water Absorbing Capabilities of the Various
Acids of half concentration in Table 7
The molality of —COOH (4.90 mol/kg)

| | | Percentage |
|---|---|---|
| | Concentration | Concentration of water in |
| | of water in | solvent mixture after |
| | wet solvent | drying with Regenerant |
| New Acid Regenerants | mixture (%) | (%) |
| Poly(acrylic acid) solution | 7.10 | 6.40 |
| glycolic acid | 7.10 | 5.00 |
| poly(acrylic acid-co- | 7.10 | 6.50 |
| maleic acid) solution | | |
| Tartaric Acid | 7.10 | 5.90 |
| Citric acid | 7.10 | 5.30 |

TABLE 10
The Water Absorbing Capabilities of the Various Acids in Table 7
The molality of —COOH (9.80 mol/kg)

| | | Percentage |
|---|---|---|
| | Concentration | Concentration of water in |
| | of water in | solvent mixture after |
| | wet solvent | drying with Regenerant |
| New Regenerants | mixture (%) | (%) |
| Poly(acrylic acid) solution | 7.10 | 5.40 |
| glycolic acid | 7.10 | 4.40 |
| poly(acrylic acid-co-maleic | 7.10 | 5.80 |
| acid) solution | | |
| Tartaric Acid | 7.10 | 4.90 |
| Citric acid | 7.10 | 4.40 |

TABLE 11
The Water Absorbing Capabilities of the Various Acids in Table 8
The molality of 0.20 mol/kg

| | | Percentage |
|---|---|---|
| | Concentration | Concentration of water in |
| | of water in | solvent mixture after |
| | wet solvent | drying with Regenerant |
| New Regenerants | mixture (%) | (%) |
| Poly(acrylic acid) solution | 7.10 | 6.40 |
| glycolic acid | 7.10 | 8.50 |
| poly(acrylic acid-co-maleic | 7.10 | 5.80 |
| acid)solution | | |
| Tartaric Acid | 7.10 | 8.80 |
| Citric acid | 7.10 | 8.10 |

The results showed that increasing the —COOH concentration also increased the water absorbing capacities Poly(acrylic acid-co-maleic acid) showed the best potential as a regenerant at a low concentration.

### Example 2—Amine Complex Cross over Experiment

This experiment was conducted to determine how much amine cross-over could be detected between the amine in the drying agent complex and the solvent mixture. The drying agent complex was tested against solvent mixtures at a wetness of 7.1%. The solvent mixture comprised a molar ratio of 1:2 TEA:MEK prepared in accordance to Preparative Example 1. Equal volumes of wet solvent mixture and drying agent were mixed and the resulting combination was vortexed for 30 seconds and then separated by the centrifuge fitted with a 130 mm diameter 4 arm swing rotor at 4000 rpm for 60 seconds. The samples were allowed to equilibrate overnight prior to testing. The results are shown in Table 12 and a gas chromatography calibration curve for ethylpiperidine is shown in FIG. 1.

TABLE 12

| Wet solvent mixture | Ethylpiperidine measured |
|---|---|
| (MEK:TEA) Sample | in the solvent mixture(ppm) |
| 7.1% | 2289 |

It can be seen that very little ethylpiperidine in ppm is crossing over into the solvent mixture meaning that the complex of [ethylpiperidine+citric acid] largely maintained its integrity as a complex throughout the passage of the complex through the (MEK:TEA) solvent mixture. Very little ethylpiperidine was measured in the solvent mixture. Had the ethylpiperidine crossed over and equilibrated with the triethylamine measurements of up to around 168,000 ppm would have been expected.

### Example 3

The drying capacity of various complexes [amine*+carboxylic acid containing compound] was tested and compared with the water recovery agents disclosed in Jessop et al. U.S. 2014/0076810.

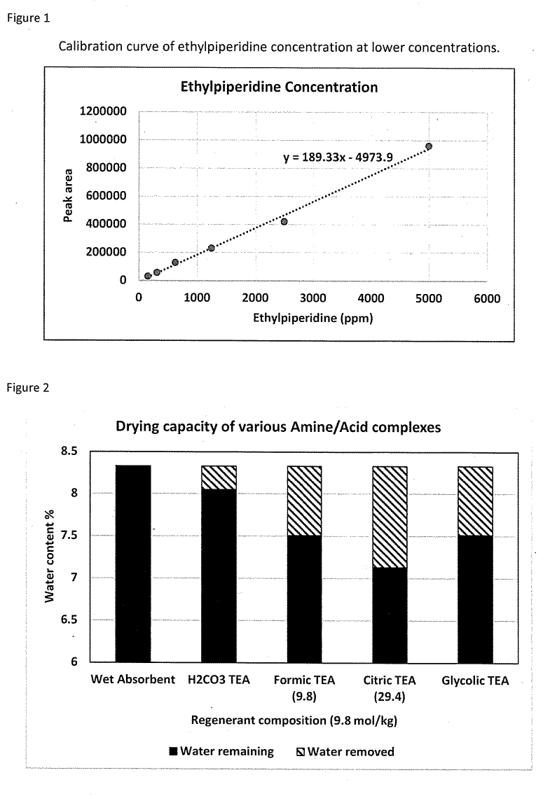

Wet solvent (TEA:MEK 1:2) prepared according to Preparative Example 1 was used and its water content was measured using gas chromatography. To 20 ml of the wet solvent mixture, 0.2 ml of the following drying agents were prepared and added to the solvent mixture and then the water content of the wet solvent mixture was remeasured using gas chromatography. TEA:CO₂ was prepared by adding TEA.H₂CO₃ (0.0098 mol, 1.60g) was added to distilled water (0.0556, 1 g). A mixture of 9.8 mol/kg of TEA:CO₂ was formed and used. TEA:Formic acid, TEA:Citric acid and TEA:Glycolic was at the same molality of 9.8 mol/kg and used to yield the results shown in Table 13 and FIG. 2.

TABLE 13

| | Water | Water |
|---|---|---|
| Drying Agent | remaining % | removed % |
| Wet solvent mixture control (no drying | 8.4 | 0 |
| agent) | | |
| TEA CO₂ (prior art) | 8.0 | 0.4 |
| TEA Formic acid (prior art) | 7.5 | 0.9 |
| TEA Citric acid | 7.1 | 1.3 |
| TEA Glycolic | 7.5 | 0.9 |

The results in Table 13 and FIG. 2 show that the triethylamine: citric acid complex and the triethylamine: glycolic acid complex provided greater or comparative water removal when compared to the system described in Jessop et al. US 2014/0076810.

### Example 4: Measurement of the pH of the Carboxylic Acid:Triethylamine Complex to Demonstrate the Irreversibilty of Protonation of the Carboxylic Acid/Triethylamine Complex

The irreversibility of the protonation of the carboxylic acid in the complex can be shown by comparing the changes in the pH showing that substantially all the free protons have been removed when triethylamine has been added—see Table 14. The pH data also supports the fact that the amine is in mostly salt form.

TABLE 14

| Citric Acid in presence | | | |
|---|---|---|---|
| of copper or iron | | | Solution |
| chloride | Triethylamine | pH | colour |
| CuCl₂ + Citric Acid | None added | 0.63 | Light blue |
| FeCl₃ + Citric Acid | None added | 0.82 | Red/brown |
| CuCl₂ + Citric Acid | TEA | 7.82 | Blue |
| FeCl₃ + Citric Acid | TEA | 7.58 | Yellow |

### Example 5: Amino Acids+Amine Combination

A range of amino acids were tested as the carboxylic group containing compounds to determine their water absorbing capacities. As above, wet solvent mixture samples were prepared according to preparative example 1 above. The amino acids were purchased from Sigma-Aldrich. The drying capacity of an amine*+various amino acid combinations were tested.

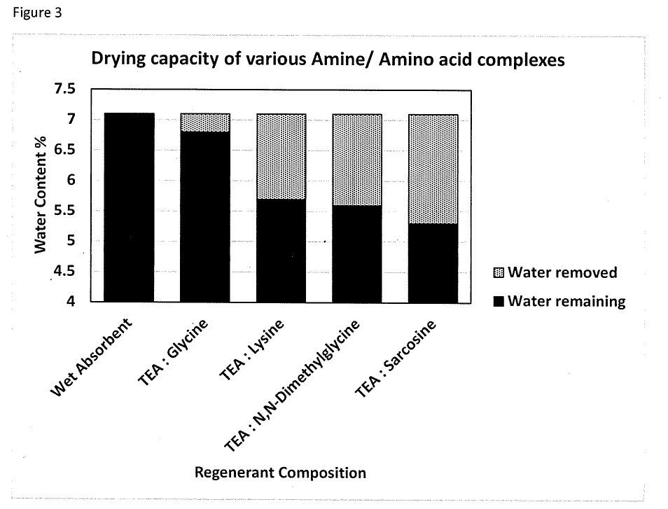

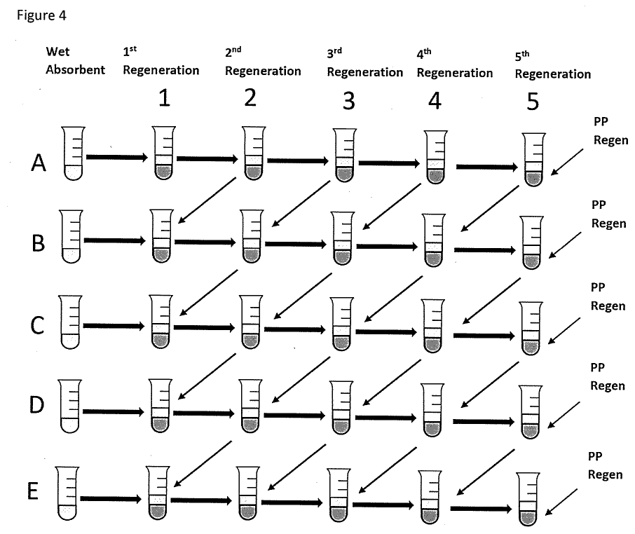

Wet solvent (TEA:MEK 1:2) prepared according to Preparative Example 1 was used and its water content was measured using gas chromatography. To 20 ml of the wet solvent mixture, 0.2 ml of the following drying agents were prepared and added to the solvent mixture and then the water content of the wet solvent mixture was remeasured using gas chromatography. Saturated amino acid solutions were mixed with TEA to form the TEA:lysine, TEA:glycine, TEA:sarcosine and TEA:N, N-dimethylglycine complexes respectively and used to yield the results shown in Table 15 and FIG. 3.

TABLE 15

| | | Percentage |
|---|---|---|
| | Percentage | Concentration |
| | Concentration | of water in solvent |
| | of water in | mixture after |
| | wet solvent | drying with |
| Drying Agent | mixture (%) | Regenerant (%) |
| TEA: Glycine | 7.10 | 6.8 |
| TEA: Lysine | 7.10 | 5.7 |
| TEA: N,N-Dimethylglycine | 7.10 | 5.6 |
| TEA: Sarcosine | 7.10 | 5.3 |

The results in Table 15 and FIG. 3 show that a triethylamine:amino acid complex can act as a drying agent. This complex is able to dry the wet solvent by effective removal of water.

### Example 6: Combinations of varying Drying Agents

A water absorbing solvent mixture was prepared according to Example 1 described above. A synthetic brine was added to the water absorbing solvent mixture in ratio of 20:1. (20 parts water absorbent solvent mixture to 1 part brine). The synthetic brine had the composition detailed in Table 16.

TABLE 16
Synthetic Brine Composition

| Salts | g/l |
|---|---|
| NaCl | 22.8 |
| MgCl₂ | 0.2348 |
| KCl | 0.1206 |
| CaCl₂ | 2.4459 |
| SrCl₂ | 0.289 |
| BaCl₂ | 0.3044 |

After the addition of the brine to the water absorbing solvent mixture, the wetness of the solvent mixture was determined by gas chromatography to be 8.136%. A series of drying agents were prepared according to Table 17.

TABLE 17
Composition of drying agents

| | | | | Moles | | Moles | Weight | Molality | Mole |
|---|---|---|---|---|---|---|---|---|---|
| | | Com- | Acid | of | Acid | of | of | of | ratio |
| | Drying | bina- | 1 | Acid | 2 | Acid | Water | —COOH | of |
| No. | Agents | tion | (g) | 1 | (g) | 2 | (g) | (mol/kg) | —COOH |
| 1 | Acid 1: | 1 | 3.6771 | 0.0245 | 4.4139 | 0.0490 | 10 | 9.8 | 1:1 |
| | Tartartic acid; | | | | | | | | |
| | Acid 2: | 2 | 2.4514 | 0.0163 | 5.8852 | 0.0653 | 10 | 9.8 | 1:2 |
| | Methoxyacetic | | | | | | | | |
| | acid | | | | | | | | |
| 2 | Acid 1: | 1 | 3.7265 | 0.0490 | 4.4139 | 0.0490 | 10 | 9.8 | 1:1 |
| | Glycolic acid; | | | | | | | | |
| | Acid 2: | 2 | 2.4843 | 0.0327 | 5.8852 | 0.0653 | 10 | 9.8 | 1:2 |
| | Methoxyacetic | | | | | | | | |
| | acid | | | | | | | | |
| 3 | Acid 1: | 1 | 3.6771 | 0.0245 | 3.1380 | 0.0163 | 10 | 9.8 | 1:1 |
| | Tartaric acid; | | | | | | | | |
| | Acid 2: | 2 | 1.8395 | 0.0123 | 4.7094 | 0.0245 | 10 | 9.8 | 1:2 |
| | citric acid | | | | | | | | |
| 4 | Acid 1: | 1 | 4.4139 | 0.0490 | 3.1380 | 0.0163 | 10 | 9.8 | 1:1 |
| | Methoxyacetic | | | | | | | | |
| | acid; | | | | | | | | |
| | Acid 2: | 2 | 2.9426 | 0.0327 | 4.1840 | 0.0218 | 10 | 9.8 | 1:2 |
| | citric acid | | | | | | | | |
| 5 | Acid1: | 1 | 3.7265 | 0.0490 | 3.6771 | 0.0245 | 10 | 9.8 | 1:1 |
| | glycolic acid; | | | | | | | | |
| | Acid2: | 2 | 1.0650 | 0.0140 | 6.3055 | 0.0420 | 10 | 9.8 | 1:2 |
| | Tartaric acid | | | | | | | | |
| 6 | Acid1: | 1 | 7.0148 | 0.0490 | 3.1380 | 0.0163 | 10 | 9.8 | 1:1 |
| | Isethionic Acid | | | | | | | | |
| | Ammonnium | | | | | | | | |
| | salt; | | | | | | | | |
| | Acid2: | 2 | 4.6766 | 0.0327 | 4.1840 | 0.0218 | 10 | 9.8 | 1:2 |
| | Citric acid | | | | | | | | |
| 7 | Acid1: | 1 | 7.1633 | 0.0490 | 3.1380 | 0.0163 | 10 | 9.8 | 1:1 |
| | Lysine; | | | | | | | | |
| | Acid2: | 2 | 3.5835 | 0.0245 | 4.7094 | 0.0245 | 10 | 9.8 | 1:2 |
| | citric acid | | | | | | | | |

0.2 ml of the drying agent prepared according to compositions 1 to 7 was added to 20 mls of the wet solvent mixture prepared above. The combination of the drying agent and the wet solvent mixture was mixed by vortex and then centrifuged to separate the respective layers. The wetness of the solvent mixture was then measured again by gas chromatography to determine how much water had been removed from the wet solvent mixture by the drying agent. The results are shown in Table 18.

TABLE 18
Viscosity, PH and conductivity of the Drying Agent

| | Drying Agent, all at 9.8 Mol/Kg of COOH and all measurements taken at 18° C. | | | | | | | |
|---|---|---|---|---|---|---|---|---|
| | Original solvent wetness was 8.136% | | | | | | | |
| | | | | | | | | Wetness |
| | | | pH | | | | | After |
| | | Mole | before | pH after | | | | Drying |
| | | ratio of | adding | adding | Viscosity | Temp | Conductivity | agent |
| Acid 1 | Acid 2 | —COOH | TEA | TEA | (m · Pas) | (° C.) | (ms/cm) | (%) |
| Tartaric | MA acid | 1:1 | 0.81 | 8.81 | 24.83 | 18 | 5.88 | 7.251 |
| acid | | 1:2 | 0.819 | 8.99 | 18.81 | | 5.53 | 7.331 |
| Glycolic | MA acid | 1:1 | 0.94 | 9.03 | 15.74 | 18 | 7.09 | 7.316 |
| acid | | 1:2 | 0.94 | 8.92 | 15 | | 6.66 | 7.285 |
| Tartaric | Citric acid | 1:1 | 0.69 | 7.86 | 154 | 18 | 4.4 | 7.346 |
| acid | | 1:2 | 0.7 | 9.14 | 63.44 | | 3.31 | 7.379 |
| MA acid | Citric acid | 1:1 | 0.97 | 8.81 | 29.73 | 18 | 4.78 | 7.447 |
| | | 1:2 | 0.86 | 8.74 | 39.94 | | 4 | 7.327 |
| Glycolic | Tartaric | 1:1 | 0.84 | 8.7 | 26.63 | 18 | 6.82 | 7.269 |
| acid | acid | 1:2 | 0.46 | 8.64 | 34.55 | | 5.09 | 7.317 |
| Lysine | Citric acid | 1:1 | 5.71 | 9.58 | 50.83 | 18 | 3.75 | 7.566 |
| | | 1:2 | 3.36 | 8.95 | 73.15 | | 3.23 | 7.479 |
| Citric acid | | | −0.31 | 8.14 | 161 | 18.3 | 1.112 | 7.298 |

MA = methoxyacetic acid

The results from Table 18 show that methoxyacetic acid provides a higher osmotic pressure in combination with tartaric acid and glycolic acid. In contrast, the osmotic pressure of the drying agents was low when the drying agent combination included lysine. It can also be seen that the viscosity of the drying agent combinations varies too. The combination of tartaric acid and citric acid has the highest viscosity.

### Example 7: Combinations of Varying Drying Agents with Different Solvent Drying Mixtures

A range of solvent drying mixtures were prepared as shown in Table 19. The molar ratio of amine to ketone was 1:2.

TABLE 19

| | | | | | Mole ratio |
|---|---|---|---|---|---|
| Ketone | | | Volume | | (Amine: |
| Amine | Mass | Density | (mL) | Mole | Ketone) |
| MEK | 74.122 | 0.806 | 171.73 | 1.87 | 0.5:1 |
| Ethylpiperidine | 113.2 | 0.824 | 128.27 | 0.93 | |
| Cyclohexanone | 98.15 | 0.948 | 180.35 | 1.74 | 0.5:1 |
| Ethylpiperidine | 113.2 | 0.824 | 119.65 | 0.87 | |
| MEK | 74.122 | 0.806 | 177.71 | 1.93 | 0.5:1 |
| 4-Ethylmorpholine | 115.1735 | 0.91 | 122.29 | 0.97 | |
| Cyclohexanone | 98.15 | 0.948 | 186.19 | 1.80 | 0.5:1 |
| 4-Ethylmorpholine | 115.1735 | 0.91 | 113.81 | 0.90 | |
| MEK | 74.122 | 0.806 | 180.92 | 1.97 | 0.5:1 |
| N,N- | 87.16 | 0.72 | 119.08 | 0.98 | |
| Diethylmethylamine | | | | | |
| Cyclohexanone | 98.15 | 0.948 | 189.32 | 1.83 | 0.5:1 |
| N,N- | 87.16 | 0.72 | 110.68 | 0.91 | |
| Diethylmethylamine | | | | | |

Gas chromatography calibrations for the solvent mixtures were prepared. These were made using 0.5, 0.49, 0.48, 0.47. 0.46 and 0.45 ml of absorbent with 0, 0.01, 0.02, 0.03, 0.04 and 0.05 ml of water respectively. The drying agents were prepared according to Table 20.

TABLE 20
Drying agents

| | Concentration | Weight of | Mole | | Number |
|---|---|---|---|---|---|
| | of COOH | Compound | of | Water | of |
| Sample | (mol/Kg) | (g) | acid | (ml) | —COOH |
| Citric acid | 9.8 | 3.138 | 0.016 | 5 | 3 |
| Glycolic | 9.8 | 3.726 | 0.049 | 5 | 1 |
| acid | | | | | |
| Tartaric | 9.8 | 3.677 | 0.025 | 5 | 2 |
| acid | | | | | |
| Lysine | 9.8 | 7.163 | 0.049 | 5 | 1 |

The ability of the ketone/amine solvent mixture to absorb water was tested in accordance with the following procedure:

10 mls of distilled water was added to 10 ml of the ketone/amine mix in a volumetric ratio of 1:1.
1 The resulting mixture was vortexed for 30 seconds and then heated to 50 degrees Celsius.
2 After 1-2 hours, the top layer of the vortexed mixture was analysed by gas chromatography.
2 The wetness of the methylethylketone and ethylpiperidine mixture was measured to be 12.6%.
4 The wetness of the cyclohexanone and ethylpiperidine mixture was measured to be 8.3%.
5 The wetness of the methylethylketone and 4-ethylmorpholine mixture was not measurable because the mixture did not separate into two phases even when heated to 70 degrees Celsius.
6 The wetness of the cyclohexanone and 4-ethylmorpholine mixture was not measurable because the mixture did not separate into two phases even when heated to 70 degrees Celsius.

The ability of a drying agent being able to release the water within the ketone/amine solvent mixture was also tested. The following drying agents were prepared by adding an excess of an amine, triethylamine (10 mis for citric acid, glycolic acid, tartaric acid and 5 ml for lysine), to the drying agent detailed in Table 20. The resulting drying agent, amine combination was then analysed for pH, viscosity and conductivity at around 19.3 degrees Celsius. The results obtained are tabulated in Table 21.

TABLE 21

| Drying | | | | | | |
|---|---|---|---|---|---|---|
| Agent | Concentration | pH before | pH after | | | |
| and | of COOH | adding | adding | Viscosity | Temperature | Conductivity |
| Amine | (mol/Kg) | TEA | TEA | (m · Pas) | (° C.) | (ms/cm) |
| Citric | 9.8 | 0.69 | 8.25 | 68.2 | 19.3 | 3.35 |
| acid•TEA | | | | | | |
| Glycolic | 9.8 | 0.84 | 9.09 | 18.7 | 19.2 | 8.27 |
| acid•TEA | | | | | | |
| Tartaric | 9.8 | 0.46 | 7.76 | 34.6 | 19.3 | 5.68 |
| acid•TEA | | | | | | |
| Lysine | 9.8 | 10.61 | 10.68 | 74.77 | 19.3 | 1.403 |
| TEA | | | | | | |

It can be observed that viscosity and conductivity were obtained from the various combinations. For example, the combination of lysine and TEA gave the highest viscosity, while the combination of glycolic acid and TEA gave the lowest viscosity. The wetness of the various solvent mixtures (ketone plus amine) with different drying agent combinations (acid plus amine) was analysed by GC and the results are shown in Table 22 below.

TABLE 22

| | | | Water | | Water | Water |
|---|---|---|---|---|---|---|
| | | Water | content of | Water | content of | content of |
| | | content of | dry ketone | content of | dry ketone | dry ketone |
| | Mole | Wet ketone | amine | dry ketone | amine | amine |
| Ketone | ratio | amine | combination | amine | combination | combination |
| Amine | (Amine: | combination | (Glycolic) | combination | (Tartaric) | (Lysine) |
| combination | Ketone) | (%) | (%) | (Citric) (%) | (%) | (%) |
| MEK | 0.5:1 | 11.931 | 10.625 | 10.526 | 10.491 | 10.81 |
| EP | | | | | | |
| CH | 0.5:1 | 7.722 | 6.842 | 6.728 | 6.744 | 7.155 |
| EP | | | | | | |
| MEK | 0.5:1 | 10.071 | 10.838 | 9.813 | 9.868 | 10.085 |
| 4-EM | | | | | | |
| CH | 0.5:1 | 9.563 | 9.814 | 9.102 | 9.224 | 9.497 |
| 4-EM | | | | | | |
| MEK | 0.5:1 | 10.905 | 10.726 | 10.267 | 10.285 | 10.42 |
| N,N-DMA | | | | | | |
| Cyclo- | 0.5:1 | 12.505 | 11.418 | 11.034 | 11.155 | 11.569 |
| hexanone | | | | | | |
| N,N-DMA | | | | | | |

EP = ethylpiperidine;
CH = cyclohexanone;
4-EM = 4-ethylmorpholine NN-DMA-N,N-diethylmethylamine

It can be seen from the results in Table 22 that the drying agents do not dry every amine: ketone solution, and notably the solutions that include 4-ethylmorpholine (EM) became wetter after mixing with a drying agent.

### Example 8—Use of Counter Current Regeneration to Optimise Recovery and Reduce Reverse Osmosis Requirements using a Commercial Brine Sample

A range of methylethylketone to triethylamine (Absorbent) mixes were prepared by adding 1 mL of a commercial brine to 20 mL of methylethylketone to triethylamine (2% wet in a MEK to TEA 1:2 ratio). The resulting sample was vortexed for 30 seconds and centrifuged for 1 min (4000 RPM). The commercial brine sample had the following composition as outlined in Table 23.

TABLE 23
Brine Sample 1 composition

| Analyte | Concentration (mg/L) |
|---|---|
| Alkalinity, Bicarbonate as CaCO₃ | 293.000 |
| Chloride | 1950.000 |
| Sulfate | 5950.000 |
| Barium | 0.012 |
| Calcium | 501.000 |
| Magnesium | 359.000 |
| Manganese | 0.011 |
| Potassium | 3.620 |
| Sodium | 3100.000 |
| Strontium | 6.930 |
| Boron | 30.700 |
| Iron | ND |
| Total Dissolved Solids | 12300.000 |

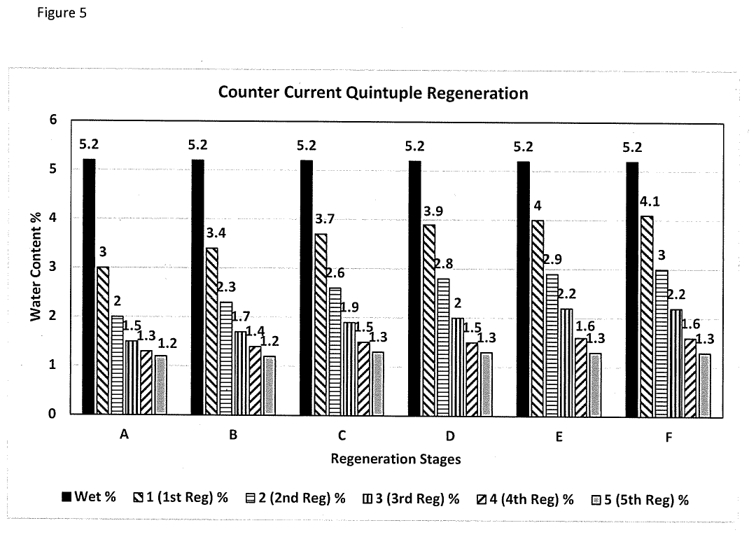

For the initial experiment A (standard Regeneration)—see FIG. 4, the Absorbent was regenerated five times with pure Regenerant (1 mL). The pure regenerant was made in a stepwise fashion using 1 litre of water, 1322 grams of citric acid, 112 grams of CuCl2 (dihydrate), 2.22 L triethylamine and 0.25 litres of methylethylketone (2 butanone).

FIG. 4 shows the steps in this experiment:
The dilute Regenerant from the 2^{nd} Regeneration is re-used for the 1^{st} Regeneration of the following stage.
The dilute Regenerant from the 3^{rd} Regeneration is re-used for the 2^{nd} Regeneration of the following stage.
The dilute Regenerant from the 4^{th} Regeneration is re-used for the 3^{rd} Regeneration of the following stage.
The 5^{th} Regeneration always used Pure Regenerant (1 mL)—denoted as PP Regen in FIG. 4.
The dilute Regenerant from the 5^{th} Regeneration is re-used for the 4th Regeneration of the next stage.

Gas chromatography analysis throughout each step for the counter current regeneration process was conducting using the parameters noted below: All GC data was collected on a SHIMADZU Nexis 2030 gas chromatograph fitted with a SH-Rxi-624Sil MS column. The GC parameters were set up as shown below:

| Parameter | Setting | |
|---|---|---|
| Injection Volume | 0.5 | μL |
| Injection temperature | 250° C. | |
| Injection mode | Split | |
| Split ratio | 50.0 | |
| Carrier gas | He | |
| Carrier gas pressure | 53.1 | kPa |
| Column flow | 1.16 | mL/min |
| Liner velocity | 24.0 | cm/s |
| Column length | 30.0 | m |
| Column inner diameter | 0.32 | |
| Column method | Gradient | |
| Column temperature | 250.0° C. | |
| Total time | 9 | min |
| Detector | TCD | |
| TCD sample rate | 40 | ms |
| TCD current | 60 | mA |
| Makeup gas | He | |
| Makeup flow | 8.0 | mL/min |

| | |
|---|---|
| TCD temperature | 200° C. |

GC Column Method:

| Rate (° C./min) | Temperature (° C.) | Hold Time (min) |
|---|---|---|
| | 100.0 | 2.00 |
| 10.00 | 125.0 | 0.00 |
| 50.00 | 200.0 | 3.00 |

Total program time 9.00 min

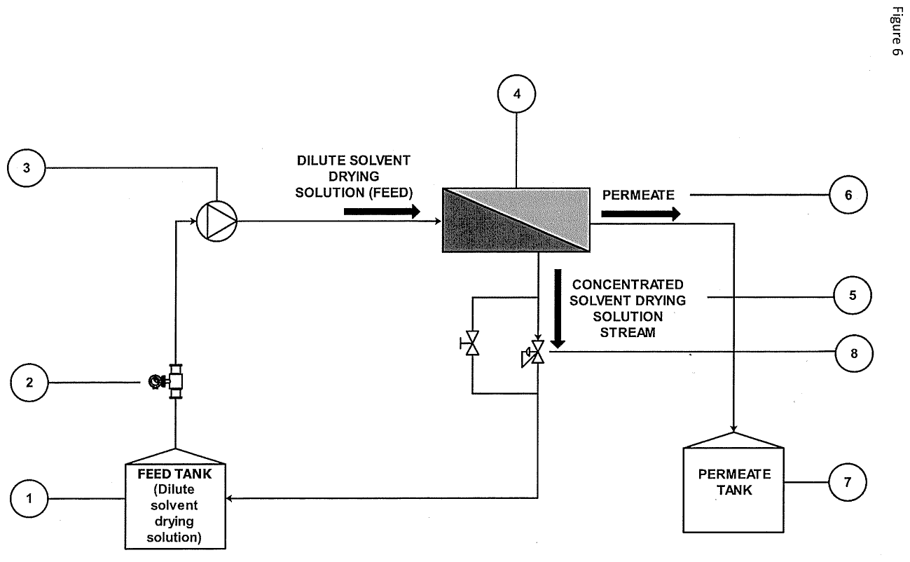

The GC analysis was conducted to determine the presence of water in the Absorbent and to track the reduction of the water levels in the Absorbent at each stage of regenerating or drying the absorbent. The GC results are shown below in Table 24 and plotted in FIG. 5.

TABLE 24

| Stages | Wet % | 1 (1^{st} Reg) % | 2 (2^{nd} Reg) % | 3 (3^{rd} Reg) % | 4 (4^{th} Reg) % | 5 (5^{th} Reg) % |
|---|---|---|---|---|---|---|
| A | 5.2 | 3.0 | 2.0 | 1.5 | 1.3 | 1.2 |
| B | 5.2 | 3.4 | 2.3 | 1.7 | 1.4 | 1.2 |
| C | 5.2 | 3.7 | 2.6 | 1.9 | 1.5 | 1.3 |
| D | 5.2 | 3.9 | 2.8 | 2.0 | 1.5 | 1.3 |
| E | 5.2 | 4.0 | 2.9 | 2.2 | 1.6 | 1.3 |
| F | 5.2 | 4.1 | 3.0 | 2.2 | 1.6 | 1.3 |

From the above results it can be seen that that after the 5th Regeneration even after re-using the Regenerant throughout all other stages the results are fairly stable and ultimately give a very low water percentage (1.3%).

The inventors have established that the water recovery performance of the Complex [amine*+carboxylic acid containing compound] is superior to the water recovery agents described in Jessop et al. U.S. 2014/0076810 as shown in Example 3. Without wanting to be bound to any mechanistic theory, it is notable that the [amine*+carboxylic acid containing compound] of the present invention is irreversibly protonated, whereas lessop et al. U.S. 2014/0076810 clearly teaches that the amine should not be irreversibly protonated. Unlike Jessop, which requires the switchability function of the drying agent, the present examples show that switchability is not a necessary function of the drying agent/regenerant. The inventors have also been able to establish that when a Complex [amine*+carboxylic acid containing compound] is mixed with a solvent mixture [amine+enolisable carbonyl+water], the amine* of the complex may be the same or different from the amine in the solvent mixture. This is because the integrity of the complex is substantially maintained as the complex passes through the solvent mixture, which is unlike what is described in Jessop. It also means that the complex or salt form of the amine is not reversible by temperature or air stripping.

### Example 9

A diluted solvent drying solution was processed using a reverse osmosis membrane. The diluted solvent drying solution (20 litres) comprised 20% by volume of the solvent drying composition and 80% by volume of distilled water. The diluted solvent drying composition was prepared by dissolving together (FeCl₃) and citric acid in the molar ratio of 1:10 and then diluting the dissolved composition with 80% of distilled water. The total dissolved solids (TDS) of the 20% (by vol.) of the solvent drying composition was approximately 287 grams. With reference to FIG. 6, the reverse osmosis system comprising the following components are illustrated:
1 Feed tank consisting of dilute solvent drying solution
2 Flow meter at the Feed outlet
3 High pressure pump with closed loop control of pressure in front of the membrane (Dow FILMTEC™ seawater reverse osmosis element SW30-2540 with active area of 2.8 m2) by manipulation of pump speed
4 Membrane vessel
5 Concentrate stream with restriction valve
6 Permeate outflow
7 Permeate collection tank
8 control valves

Prior to use of the osmosis system shown in FIG. 6, the membrane in the membrane vessel 4 was conditioned by running deionised water through the membrane for 2 hours before dosing the feed with the diluted solvent drying solution. The diluted solvent drying solution from the feed tank 1 was pushed to the high-pressure level using a high-pressure pump 3. The semi-permeable membrane inside of each membrane vessel 4 restrains most of the solvent drying composition. Only the permeate consisting of low dissolved salt and water gets through the membrane, while the concentrate stream 5 is fed back into the feed tank 1. The permeate outflow 6 is fed into the permeate collection tank 7. The electrical conductivity of the permeate was measured as an indicator of permeate quality and rejection %.

Measurement Conditions:
Max. operating temperature: 40° C.
Max. membrane operating temperature: 45° C.
Pressure (bar): 60
The permeate flow rate and conductivity measurements of both concentrate and permeate were collected at the below mentioned time intervals.

TABLE 25
Feed: 20% (by vol.) diluted solvent drying solution

| | Time | Flux(LMH) | Rejection % |
|---|---|---|---|
| 60 bar | 2 | 3.51 | 97.33 |
| | 4 | 3.58 | 98.22 |
| | 6 | 3.74 | 98.32 |
| | 8 | 3.56 | 98.25 |
| | 10 | 3.45 | 98.13 |
| | 12 | 3.25 | 97.93 |
| | 14 | 2.98 | 97.71 |
| | 16 | 2.88 | 97.38 |
| | 18 | 2.74 | 97.04 |
| | 20 | 2.68 | 97.01 |
| | 30 | 1.80 | 94.30 |
| | 40 | 1.75 | 93.77 |
| | 50 | 1.63 | 93.68 |
| | 60 | 0.65 | 93.00 |
| | 70 | 0.09 | 93.03 |

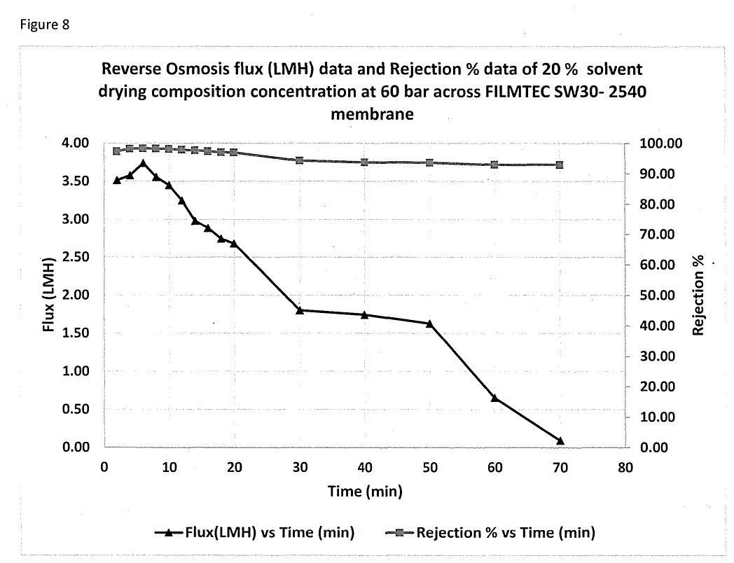

The results shown in Table 25 are also shown plotted in FIG. 8.

Osmotic pressure and concentration measurements: 100 uL of sample was taken from both feed and permeate and run through the Osmometer. The units were converted from mOsmol/kg to atm and the concentration of salt in both the streams was calculated and tabulated.

The following formulae were used to calculate flux, salt rejection and water recovery.

Flux Measurement:

$$Jw = \frac{Flow rate of Permeate ( \frac{mL}{min} ) \times 60}{1000 \times Membrane active area}$$

Salt Rejection % by Conductivity Method:

$$\frac{\begin{matrix}Conductivity of feed solution ( \frac{mS}{cm} ) - \\ Conductivity of Permeate water ( \frac{mS}{cm} )\end{matrix}}{Conductivity of feed solution ( \frac{mS}{cm} )} \times 100$$

Salt Rejection % by Osmotic Pressure Method:

$$\frac{\begin{matrix}Osmotic pressure of feed ( atm ) - \\ Osmotic pressure of permeate ( atm )\end{matrix}}{Osmotic pressure of feed ( atm )} \times 100$$

Water Recovery %—Method 1:

$$\frac{Volume of ( Feed - Concentrate ) ( L )}{Volume of feed ( L )} \times 100$$

Water Recovery %—Method 2:

$$\frac{Volume of permeate collected at end of test ( L )}{Volume of feed ( L )} \times 100$$

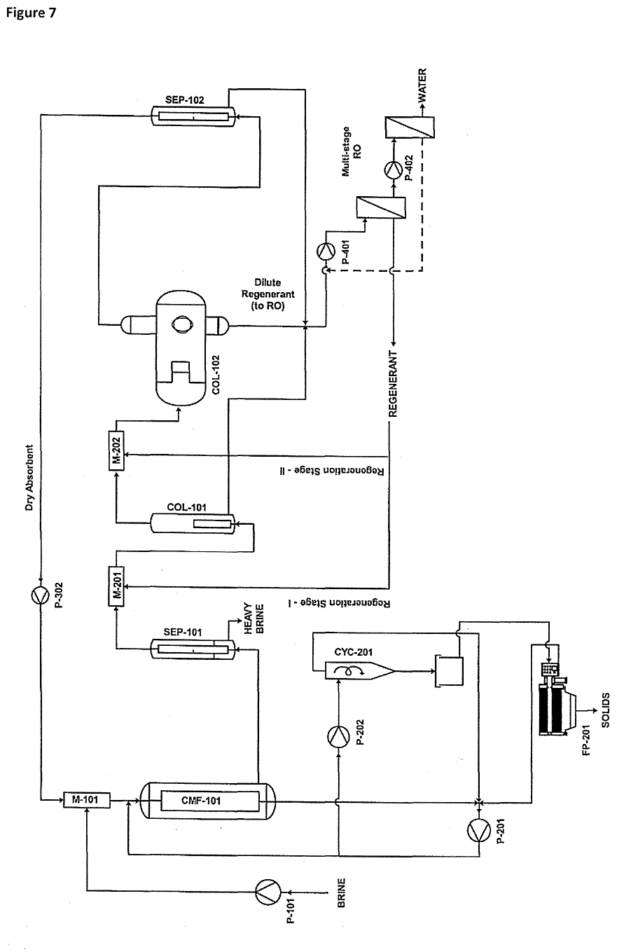

A second embodiment of the process of the present invention is shown in FIG. 7. This embodiment illustrates a process where more than one solvent drying composition regeneration step may be employed to recover the solvent drying composition complex. As shown in FIG. 7, the diluted regenerant (the dilute solvent drying composition) is recovered from the coalescer column COL-102 after an industrial process involving the removal of water from a brine feed stock. The diluted solvent drying composition is then subjected to a multi-stage reverse osmosis recovery phase to concentrate (ie remove water) the solvent drying composition (regenerant) in a continuous loop operation, whereby the regenerant is recovered and then fed back into the earlier stages of the industrial process to facilitate the removal of water from a brine solution. It is to be appreciated that the coalescer column may be an electrostatic coalescer column, because the solvent drying composition is a good insulator and electrostatic coalescing may improve the overall performance of the process. FIG. 11 shows a process diagram that includes an electrostatic coalscer (COL-202).

### Example 10—Other Membranes

Various other membranes were also tested under the following conditions and compared to the membrane used above:

Solvent Drying Composition—The diluted solvent drying composition was prepared by dissolving together (FeCl3) and citric acid in the molar ratio of 1: 10 and then diluting the dissolved composition with 80% of distilled water. The total dissolved solids (TDS) of the 20% (by vol.) of the solvent drying composition was approximately 287 grams.

### Example 10.1 Membrane 1 TriSep™ TS-80

Membrane Specifications:
Flux (GFD/psi): 220/110
Max. operating pressure (bar): 41
Max. operating temperature (° C.): 45
Chlorine tolerance: 0.1 ppm
Membrane active area: 0.0142 m²
Feed solution: 5% Solvent Drying solution (by vol.)

Membrane 1 Results:

The results at various pressures and times for flux and salt rejection data are shown below in Tables 26-28.

TABLE 26
Flux (LMH) and salt rejection % data for TriSep ™ TS-80

| | Permeate Flux | |
|---|---|---|
| Pressure (bar) | (LMH) | Rejection % |
| 20 | 11.15 | 59.09 |
| 25 | 11.59 | 59.97 |
| 30 | 14.46 | 60.47 |
| 35 | 17.71 | 60.31 |

TABLE 27
Flux (LMH) at different pressures at regular
time interval for TriSep ™ TS-80

| Pressure (bar) | 20 | 25 | 30 | 35 |
|---|---|---|---|---|
| Time | Flux | Flux | Flux | Flux |
| (min) | (LMH) | (LMH) | (LMH) | (LMH) |
| 5 | 10.72 | 11.04 | 15.41 | 18.10 |
| 10 | 11.74 | 12.51 | 13.53 | 16.56 |
| 15 | 10.50 | 11.55 | 14.82 | 18.02 |
| 20 | 11.38 | 11.74 | 14.44 | 18.54 |
| 25 | 11.10 | 11.18 | 14.59 | 17.32 |
| 30 | 11.48 | 11.55 | 13.96 | 17.74 |

Osmotic Pressure Data

TABLE 28
Calculating rejection % using Osmotic pressure
measurements of feed and permeate streams

| | Osmotic | Osmotic | |
|---|---|---|---|
| Pressure | pressure of | pressure of | Reduction |
| (bar) | feed (bar) | permeate (bar) | % |
| 20.00 | 2.71 | 1.22 | 54.79 |
| 25.00 | 2.68 | 1.16 | 56.80 |
| 30.00 | 2.74 | 1.20 | 56.21 |
| 35.00 | 2.67 | 1.24 | 53.64 |

### Example 10.2 Membrane 2 Dow Filmtec Flat Sheet Membrane, SW30XLE, PA-TFC, RO

Membrane Specifications:
Flux (GFD/psi): 23-29/880
Max. operating pressure (bar): 68.9
Max. operating temperature (° C.): 45
Chlorine tolerance: 0.1 ppm
Membrane active area: 0.0142 m²
Feed solution: 5% Solvent Drying solution (by vol.)

Membrane 2 Results:

The results at various pressures and times for flux and salt rejection data are shown below in Tables 28-30.

TABLE 29
Flux (LMH) and salt rejection % data

| Pressure (bar) | Permeate Flux (LMH) | Rejection % |
|---|---|---|
| 20 | 7.08 | 62.48 |
| 25 | 8.82 | 69.39 |
| 30 | 11.09 | 80.25 |
| 35 | 14.98 | 86.79 |
| 40 | 18.49 | 88.57 |

TABLE 30
Flux (LMH) at different pressures
at regular time intervals

| Pressure (bar) | 20 | 25 | 30 | 35 | 40 |
|---|---|---|---|---|---|
| Time | Flux | Flux | Flux | Flux | Flux |
| (min) | (LMH) | (LMH) | (LMH) | (LMH) | (LMH) |
| 5 | 7.10 | 9.30 | 11.26 | 13.98 | 17.59 |
| 10 | 7.18 | 8.45 | 11.21 | 16.74 | 18.34 |
| 15 | 6.87 | 8.41 | 11.26 | 14.90 | 18.11 |
| 20 | 7.24 | 8.54 | 11.04 | 14.56 | 18.97 |
| 25 | 6.84 | 9.35 | 11.21 | 14.73 | 18.68 |
| 30 | 7.28 | 8.86 | 10.59 | 14.97 | 19.27 |

Osmotic Pressure Data

TABLE 31
Calculating rejection % using Osmotic pressure
measurements of feed and permeate streams

| | | Measured | |
|---|---|---|---|
| | Measured | osmotic | |
| | osmotic | pressure | |
| Pressure | pressure of | of permeate | Reduction |
| (bar) | feed (bar) | (bar) | % |
| 20.00 | 2.49 | 0.67 | 72.96 |
| 25.00 | 2.82 | 0.47 | 83.33 |
| 30.00 | 2.67 | 0.37 | 86.02 |
| 35.00 | 2.71 | 0.21 | 92.24 |
| 40.00 | 2.68 | 0.19 | 93.05 |

### Example 10.3 Membrane 3 Toray Flat Sheet Membrane—UTC-82V, PA, RO

Membrane Specifications:
Flux (GFD/psi): 27/798
Max. operating pressure (bar): 55
Max. operating temperature (° C.): 25
Membrane active area: 0.0142 m²
Feed solution: 5% Solvent Drying solution (by vol.)

Membrane 3 Results:

The results at various pressures and times for flux and salt rejection data are shown below in Tables 32-34.

TABLE 32
Flux (LMH) and salt rejection % data

| Pressure (bar) | Permeate Flux (LMH) | Rejection % |
|---|---|---|
| 20 | 23.84 | 67.11 |
| 25 | 27.73 | 76.30 |
| 30 | 29.67 | 75.17 |
| 35 | 34.30 | 78.22 |
| 40 | 38.96 | 81.86 |
| 45 | 48.59 | 85.65 |

TABLE 33
Flux (LMH) at different pressures
at regular time intervals

| Pressure (bar) | 20 | 25 | 30 | 35 | 40 |
|---|---|---|---|---|---|
| Time | Flux | Flux | Flux | Flux | Flux |
| (min) | (LMH) | (LMH) | (LMH) | (LMH) | (LMH) |
| 5 | 25.18 | 27.65 | 30.36 | 35.88 | 38.70 |
| 10 | 23.41 | 28.21 | 29.80 | 35.95 | 38.28 |
| 15 | 23.88 | 27.34 | 29.87 | 33.18 | 38.79 |
| 20 | 23.73 | 27.37 | 30.22 | 33.33 | 38.45 |
| 25 | 23.49 | 27.30 | 29.54 | 34.90 | 38.70 |
| 30 | 23.32 | 28.53 | 28.25 | 32.54 | 40.82 |

Osmotic Pressure Calculations:

TABLE 34
Calculating rejection % using Osmotic pressure
measurements of feed and permeate streams

| | Measured | Measured | |
|---|---|---|---|
| | osmotic | osmotic | |
| | pressure | pressure of | |
| Pressure | of feed | permeate | Reduction |
| (bar) | (bar) | (bar) | % |
| 20.00 | 3.78 | 0.96 | 74.73 |
| 25.00 | 3.35 | 0.78 | 76.76 |
| 30.00 | 3.48 | 0.71 | 79.72 |
| 35.00 | 3.41 | 0.61 | 82.19 |
| 40.00 | 3.55 | 0.52 | 85.39 |
| 45.00 | 3.68 | 0.45 | 87.67 |

### Example 10.4 Membrane 4 Synder Flat Sheet Membrane, NFX, PA-TFC, NF

Membrane Specifications:

### Flux (GFD/psi): 20-25/110

Max. operating pressure (bar): 30
Max. operating temperature (° C.): 35
Chlorine tolerance (ppm hours): 500
Membrane active area: 0.0142 m²
Feed solution: 5% Solvent Drying solution (by vol.)

Membrane 4 Results:

The results at various pressures and times for flux and salt rejection data are shown below in Tables 35-37.

TABLE 35
Flux (LMH) and salt rejection % data

| Pressure | Permeate Flux | Rejection |
|---|---|---|
| (bar) | (LMH) | % |
| 20 | 40.49 | 56.55 |
| 25 | 47.45 | 54.67 |
| 30 | 59.05 | 52.42 |
| 35 | 65.62 | 53.39 |

TABLE 36
Flux (LMH) at different pressures
at regular time intervals

| Pressure (bar) | 20 | 25 | 30 | 35 |
|---|---|---|---|---|
| Time | Flux | Flux | Flux | Flux |
| (min) | (LMH) | (LMH) | (LMH) | (LMH) |
| 5 | 42.81 | 44.20 | 55.79 | 63.78 |
| 10 | 45.63 | 45.21 | 54.29 | 67.38 |
| 15 | 42.69 | 49.94 | 59.36 | 67.25 |
| 20 | 35.56 | 49.94 | 63.56 | 60.23 |
| 25 | 38.45 | 48.21 | 60.41 | 68.34 |
| 30 | 37.77 | 47.17 | 60.87 | 66.76 |

Osmotic Pressure Calculations:

TABLE 37
Calculating rejection % using Osmotic pressure
measurements of feed and permeate streams

| | Measured | Measured | |
|---|---|---|---|
| | osmotic | osmotic | |
| | pressure | pressure of | |
| Pressure | of feed | permeate | Reduction |
| (bar) | (bar) | (bar) | % |
| 20 | 3.48 | 0.95 | 72.73 |
| 25 | 3.20 | 0.80 | 74.90 |
| 30 | 3.10 | 0.69 | 77.65 |
| 35 | 3.26 | 0.97 | 70.15 |

### Example 10.5 Dow Filmtec Flat Sheet Membrane, SW30HR, PA-TFC, RO Membrane

Membrane Specifications:
Flux (GFD/psi): 18-24/800
Max. operating pressure (bar): 68.9
Max. operating temperature (° C.): 45
Chlorine tolerance (ppm hours): 0.1
Membrane active area: 0.0142 m²
Feed solution: 5% Solvent Drying solution (by vol.)

Results:

The results at various pressures and times for flux and salt rejection data are shown below in Tables 38-40.

TABLE 38
Flux (LMH) and salt rejection % data

| Pressure | Permeate Flux | Rejection |
|---|---|---|
| (bar) | (LMH) | % |
| 35 | 7.74 | 84.78 |
| 40 | 13.18 | 93.32 |

TABLE 39
Flux (LMH) at different pressures
at regular time intervals

| Pressure (bar) | 35 | 40 |
|---|---|---|
| Time (min) | Flux (LMH) | Flux (LMH) |
| 5 | 7.43 | 12.08 |
| 10 | 7.65 | 20.79 |
| 15 | 7.75 | 11.58 |
| 20 | 7.47 | 11.47 |
| 25 | 7.93 | 11.65 |
| 30 | 8.23 | 11.51 |

Osmotic Pressure Calculations:

TABLE 40
Calculating rejection % using Osmotic pressure
measurements of feed and permeate streams

| | Measured | Measured | |
|---|---|---|---|
| | osmotic | osmotic | |
| | pressure | pressure of | |
| Pressure | of feed | permeate | Reduction |
| (bar) | (bar) | (bar) | % |
| 35 | 3.52 | 0.24312 | 93.09 |
| 40 | 3.43 | 0.08104 | 97.64 |
| 45 | 3.64 | 0.072936 | 98.00 |

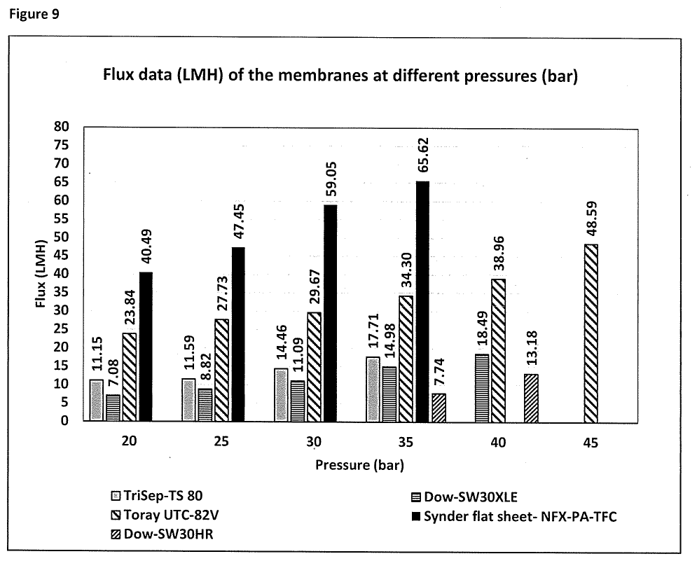

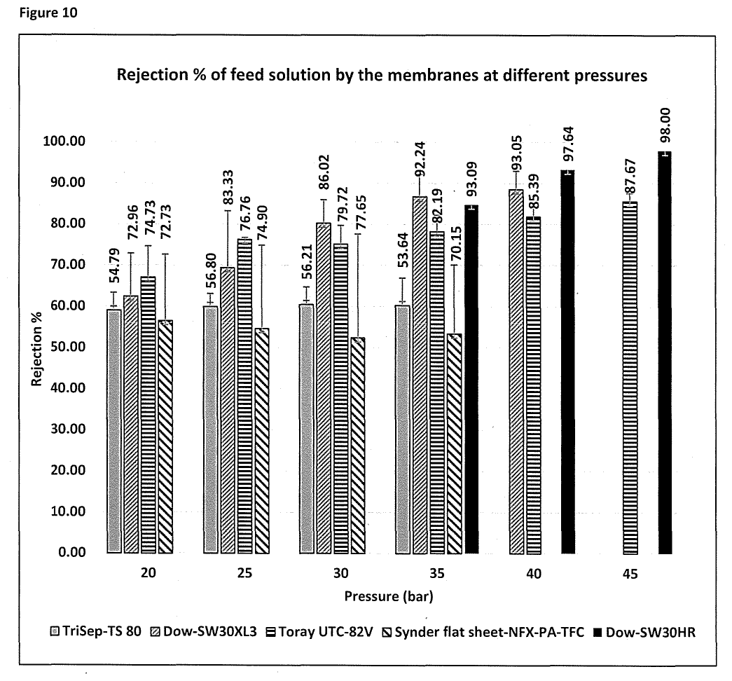

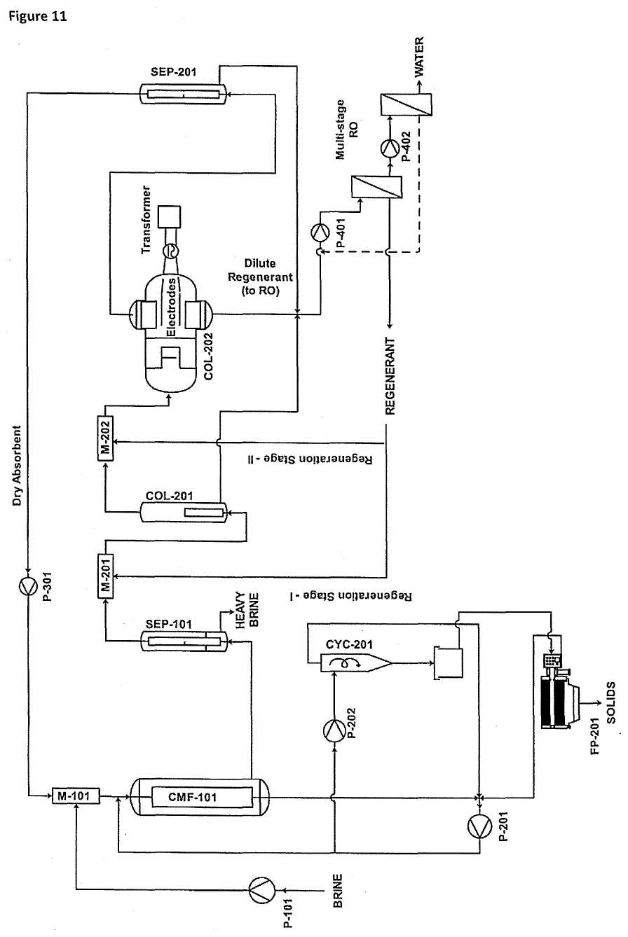

The results of the various membranes are shown in FIGS. 9 and 10. It can be seen from FIGS. 9 and 10 that the results of the membranes are able to recover water from the dilute solvent drying solution using a range of commercially available membranes.

### Example 11: Determination of Whether Different Metal Salts Affect the Water Capacity of the Solvent Drying Composition

A range of solvent drying compositions were prepared with different metal salts and their respective water capacities were determined by gas chromatography. The solvent drying compositions were prepared as follows:
1. A quantity of a specific metal salt (detailed in Table 41 below) was added to a solution of citric acid (6.6gm or 0.340 mol) in distilled water (5 ml).
2. The resulting mixture was stirred at 80 degrees Celsius for 20 minutes.
3. Excess triethylamine was added to the stirred mixture from step 2 to generate the solvent drying composition.

TABLE 41

| | Metal | Moles of | Weight | Moles | | |
|---|---|---|---|---|---|---|
| Metal | salt | Metal | of citric | of citric | distilled | Mole ratio |
| salt | weight | Salt | acid | acid | water | (X:10 citric |
| used | (g) | (mol) | (g) | (mol) | (g) | acid) |
| NaCl | 0.2008 | 0.0034 | 6.6 | 0.0344 | 5 | 1 |
| Na₂CO₃ | 0.3641 | 0.0034 | 6.6 | 0.0344 | 5 | 1 |
| SrCl₂ | 0.5445 | 0.0034 | 6.6 | 0.0344 | 5 | 1 |
| AlCl₃ | 0.8295 | 0.0034 | 6.6 | 0.0344 | 5 | 1 |
| FeCl₃ | 0.5572 | 0.0034 | 6.6 | 0.0344 | 5 | 1 |
| CuCl₂ | 0.5857 | 0.0034 | 6.6 | 0.0344 | 5 | 1 |
| Fe(NO₃)₃ | 1.3879 | 0.0034 | 6.6 | 0.0344 | 5 | 1 |
| Fe2(SO₄)₃ | 1.3737 | 0.0034 | 6.6 | 0.0344 | 5 | 1 |
| CuSO₄ | 0.5483 | 0.0034 | 6.6 | 0.0344 | 5 | 1 |
| Cu(OH)₂ | 0.3400 | 0.0034 | 6.6 | 0.0344 | 5 | 1 |

The properties of the solvent drying compositions prepared are detailed in Table 42 below:

TABLE 42

| | pH | pH | | | Osmotic | |
|---|---|---|---|---|---|---|
| Solvent Drying | before adding | after adding | Viscosity | Temp | Pressure | Conductivity |
| Composition | TEA | TEA | (m · Pas) | (° C.) | (atm) | (ms/cm) |
| NaCl•citrate | 0.04 | 8.05 | 288 | 18 | 194 | 1.119 |
| Na₂CO₃•citrate | 1.58 | 8.09 | 375 | 18 | 186 | 1.182 |
| SrCl₂•citrate | 1.27 | 8.18 | 381.32 | 18 | 194 | 0.949 |
| AlCl₃•citrate | −0.43 | 8.29 | 209.22 | 18 | — | 1.569 |
| FeCl₃•citrate | −0.28 | 8.06 | 219.68 | 18 | 186 | 1.96 |
| CuCl₂•citrate | 0.17 | 8.13 | 238.36 | 18 | 158 | 1.559 |
| Fe(NO₃)₃•citrate | −0.24 | 8.22 | 166.18 | 18 | — | 2.2 |
| Fe2(SO₄)₃•citrate | −0.14 | 8.25 | 274.14 | 18 | 182 | 1.111 |
| CuSO₄•citrate | 0.04 | 8.16 | 278 | 18 | — | 1.258 |
| Cu(OH)₂•citrate | 0.98 | 8.016 | 537.23 | 18 | 108 | 1.005 |

It can be seen that the viscosity of each solvent drying composition varies as the metal salt changes. The above solvent drying compositions were then reacted with wet absorbent as follows:
1. 0.2 ml of each solvent drying composition outlined in Table X was added to 20 ml of wet absorbent.
2. The resulting mixture was mixed by vortex mixer for 30 seconds and then separated by centrifuge.
3. A GC analysis of the water remaining in the absorbent after mixing with the solvent drying compositions was analysed and the results shown in Table 43 below.

TABLE 43
The water absorption capacities of Regenerants

| | | | | Water | |
|---|---|---|---|---|---|
| | | | | content in | |
| | | | | Absorbent | |
| | | | | after | |
| | | | Volume | drying | |
| | | Water | of | with | |
| | Wet | content | Solvent | Solvent | |
| | Ab- | in wet | Drying | Drying | |
| Solvent | sorbent | Ab- | Compo- | Compo- | |
| Drying | used | sorbent | sition | sition | Valence |
| Composition | (mL) | (%) | (mL) | (%) | state |
| NaCl•citrate | 20 | 7.793 | 0.2 | 6.635 | 1 |
| Na₂CO₃• | 20 | 7.793 | 0.2 | 6.522 | 1 |
| citrate | | | | | |
| SrCl₂•citrate | 20 | 7.793 | 0.2 | 6.581 | 2 |
| AlCl₃•citrate | 20 | 7.793 | 0.2 | 6.778 | 3 |
| FeCl₃•citrate | 20 | 7.793 | 0.2 | 6.76 | 3 |
| CuCl₂• | 20 | 7.793 | 0.2 | 6.802 | 2 |
| citrate | | | | | |
| Fe(NO₃)₃• | 20 | 7.793 | 0.2 | 6.834 | 3 |
| citrate | | | | | |
| Fe2(SO₄)₃• | 20 | 7.793 | 0.2 | 6.634 | 3 |
| citrate | | | | | |
| CuSO₄• | 20 | 7.793 | 0.2 | 6.723 | 2 |
| citrate | | | | | |
| Cu(OH)₂• | 20 | 7.793 | 0.2 | 6.699 | 2 |
| citrate | | | | | |

It can be seen from the results shown in Table 43 that the water absorption capacity of each solvent drying composition is not substantially altered as the metal salt changes.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to combinations, kits, compounds, means, methods, and/or steps disclosed herein.

1. A solvent drying composition, the composition comprising of:
a) a complex of at least one amine or ammonium salt containing compound and at least one carboxylic acid containing compound or an alkylsulfonic acid; or a combination thereof, in a solvent comprising
b) at least one amine containing compound at least one enolisable carbonyl and water, wherein in use water in the solvent is released to form an immiscible aqueous layer with the solvent drying composition.

2. The composition as claimed in claim 1 wherein the carboxylic acid containing compound is selected from one or more of the following:
a) compound of Formula I, 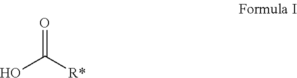
i. wherein R* is selected from, -C₁-C₇ alkyl-OH, -C₁-C₇ alkyl, -C₁-C₇ alkyl-NH₂, -C₁-C₇ alkyl-NHR³ and -C₁-C₇ alkyl NR³R⁴, wherein each R³ and R⁴ are selected from —H, —OH, -halo, -C₁-C₇ alkyl, -C₁-C₇ alkyl-OH, —C(O)OH, —C(O)—H, or —C(O)-(C₁-C₇ alkyl); or
ii. wherein the compound of Formula I is acetic acid, citric acid and glycolic acid or a combination thereof;
b) a polymer containing one or more carboxylic acid groups.

3. The composition as claimed in claim 1, wherein the solvent from which the water is recovered comprises:
(a) at least one amine containing compound, or
(b) at least a secondary or tertiary amine containing compound or a combination thereof; and
(c) at least one enolisable carbonyl, or
(d) at least one enolisable carbonyl of Formula II.

4.-6. (canceled)

7. The composition as claimed in claim 1, wherein the alkylsulfonic acid is isoethionic acid.

8. The composition as claimed in claim 1, wherein the molar ratio of the at least one amine or ammonium salt containing compound to the at least one carboxylic acid containing compound or an alkylsulfonic acid; or a combination thereof, is selected from
(a) about 1:99 or 99:1;
(b) about 1:50 or 50:1;
(c) about 1:10 or 10:1;
(d) about 1:5 or 5:1;
(e) about 1:3 or 3:1;
(f) about 1:2 or 2:1; and
(g) about 1:1.

9.-14. (canceled)

15. The composition as claimed in claim 1, wherein the solvent includes at least one amine containing compound, which amine may be the same or different from the amine containing compound in the complex; and wherein the at least one amine containing compound of the complex or solvent is selected from a conjugated, aliphatic, asymmetric or cyclic tertiary amine.

16. (canceled)

17. The composition as claimed in claim 15, wherein the tertiary amine containing compound is selected from the following:
(a) 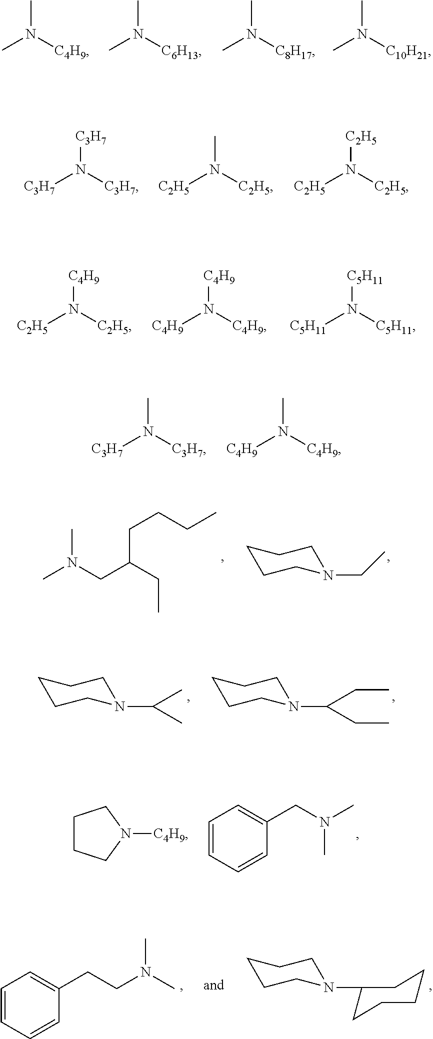
or
(b) N(C₁-C₇ alkyl)₃, N(C₁-C₄ alkyl)₃ N(C₂ alkyl)₃ (triethylamine) or ethylpiperidine.

18.-23. (canceled)

24. The composition as claimed in claim 3 wherein Formula II, is 2-butanone.

25. The composition as claimed in claim 3, wherein the molar ratio of the at least one tertiary amine containing compound to the one or more enolisable carbonyls of Formula II are present in a ratio of:
(a) about 1:99 or 99:1;
(b) about 1:50 or 50:1;
(c) about 1:10 or 10:1;
(d) about 1:5 or 5:1;
(e) about 1:3 or 3:1;
(f) about 1:2 or 2:1; or
(g) about 1:1.

26.-31. (canceled)

32. The composition as claimed in claim 1, wherein the at least one amine or ammonium salt containing compound and at least one carboxylic acid containing compound or an alkylsulfonic acid; or a combination thereof, is irreversibly protonated.

33. A complex composition comprising:
a. at least one amine or ammonium salt containing compound or a secondary or tertiary amine containing compound or a combination thereof; and
b. at least an alkylsulfonic acid; or at least one carboxylic acid containing compound of Formula I; or a combination thereof; 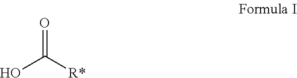
(i) wherein R* is selected from, -C₁-C₇ alkyl-OH, -C₁-C₇ alkyl, -C₁-C₇ alkyl-NH₂, -C₁-C₇ alkyl-NHR³ and -C₁-C₇ alkyl NR³R⁴, wherein each R³ and R⁴ are selected from —H, —OH, -halo, -C₁-C₇ alkyl, -C₁-C₇ alkyl-OH, —C(O)OH, —C(O)—H, or —C(O)-(C₁-C₇ alkyl)
(ii) wherein the compound of Formula I is acetic acid, citric acid and glycolic acid or a combination thereof;
the complex being suitable for use in recovering water from a solvent, wherein water is released from the solvent upon migration of the composition through the solvent, the released water forming an immiscible aqueous layer with the solvent
and wherein the solvent comprises:
a) at least one amine containing compound or at least one secondary or tertiary amine containing compound,
b) at least one enolisable carbonyl, and
c) water.

34. (canceled)

35. The complex as claimed in claim 33, wherein the solvent comprises at least one enolisable carbonyl of Formula II 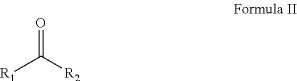
wherein
a) R₁ and R₂ are independently selected from a -C₁-C₇ alkyl or a -C₃-C₇ monocyclic or a phenyl; or
b) one of R₁ or R₂ is selected from a —O-(C₁-C₇ alkyl) and the other is selected from a -C₁-C₇ alkyl, or
c) R₁ and R₂ together, with the carbonyl of Formula I, form a 3-15 membered monocyclic ketone or a 3-15 membered monocyclic heterocyclic ketone or acetephenone;
wherein if R₁ is -C1-C7 alkyl, R₁ may be optionally substituted with one or more substituents selected from —OH, -C1-C7 alkyl, -(C1-C7 alkyl)-OH, —NH2, —NH(C1-C7 alkyl), —N(C1-C7 alkyl)2, —C(O)OH; —C(O)—H, or —C(O)-(C1-C7 alkyl).

36. (canceled)

37. The complex as claimed in claim 33, wherein the carboxylic acid containing compound of Formula I is selected from acetic acid, citric acid and glycolic acid or a combination thereof.

38. The complex as claimed in claim 33, wherein the alkylsulfonic acid is isoethionic acid.

39. The complex as claimed in claim 33, wherein the molar ratio of the at least one amine or ammonium salt containing compound to the at least one carboxylic acid containing compound or an alkylsulfonic acid; or a combination thereof, is
(a) about 1:99 or 99:1;
(b) about 1:50 or 50:1;
(c) about 1:10 or 10:1;
(d) about 1:5 or 5:1;
(e) about 1:3 or 3:1;
(f) about 1:2 or 2:1; and
(g) about 1:1.

40.-45. (canceled)

46. The complex as claimed in claim 33, wherein the solvent includes at least one amine containing compound, which amine may be the same or different from the amine containing compound in the complex; and wherein the at least one amine containing compound of the complex or solvent is selected from a conjugated, aliphatic, asymmetric or cyclic tertiary amine.

47. (canceled)

48. The complex as claimed in claim 46, wherein the tertiary amine containing compound is selected from the following:
a) 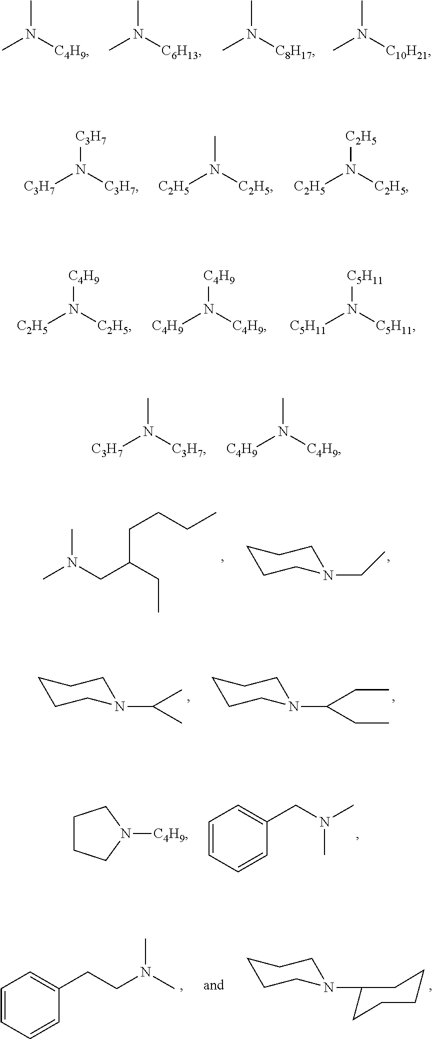
or
b) N(C₁-C₇ alkyl)₃, N(C₁-C₄ alkyl)₃, N(C₂ alkyl)₃ (triethylamine) or ethylpiperidine.

49.-55. (canceled)

56. The complex as claimed in claim 35, wherein Formula II, is 2-butanone.

57. The complex as claimed in claim 33, wherein the molar ratio of the at least one tertiary amine containing compound to the one or more enolisable carbonyls of Formula II are present in a ratio of:
(a) about 1:99 or 99:1;
(b) about 1:50 or 50:1;
(c) about 1:10 or 10:1;
(d) about 1:5 or 5:1;
(e) about 1:3 or 3:1;
(f) about 1:2 or 2:1; and
(g) about 1:1.

58.-63. (canceled)

64. The complex as claimed in claim 33, wherein the at least one amine or ammonium salt containing compound and at least one carboxylic acid containing compound or an alkylsulfonic acid; or a combination thereof, is irreversibly protonated.

65. A method of recovering water from a solvent, the method including the steps of:
a) contacting the solvent with the solvent drying composition of claim 1 for use in recovering water from a solvent, claim 1, or
b) contacting the solvent with a complex composition as claimed in claim 33; and
c) allowing the migration of the solvent drying composition or the complex composition through the solvent, whereupon the water is released from the solvent forming an immiscible aqueous layer with the solvent.

66. The method of claim 65, wherein the method includes the step of separating the recovered water for the immiscible solvent layer.

67-69. (canceled)

70. The method as claimed in claim 65, wherein the solvent is contacted with one or more solvent drying compositions or one or more complex compositions iteratively to iteratively release water therefrom.

71. The method as claimed in claim 70, wherein the solvent is contacted with one or more solvent drying compositions or one or more complex compositions iteratively in a counter-current process.

72.-120. (canceled)
